(19) United States
(12) Patent Application Publication
(10) Pub. No.: US 2024/0352136 A1
(43) Pub. Date: Oct. 24, 2024

(54) ANTIBODIES FOR THE TREATMENT OF AML

(71) Applicants: Hoffmann-La Roche Inc., Little Falls, NJ (US); Genentech, Inc., South San Francisco, CA (US)

(72) Inventors: Maria Amann, Duebendorf (CH); Laurène Pousse, Zurich (CH); Christophe Boetsch, Colmar (FR); Martin Weisser, Penzberg (DE); Theresa Kolben, Munich (DE); Vaios Karanikas, Zurich (CH); Jan Eckmann, Munich (DE); Bruno Carneiro de Medeiros, San Francisco, CA (US)

(21) Appl. No.: 18/687,561

(22) PCT Filed: Sep. 2, 2022
(86) PCT No.: PCT/EP2022/074455

§ 371 Date: Feb. 28, 2024

Related U.S. Application Data
(60) Provisional application No. 63/240,342, filed on Sep. 2, 2021.

(51) Int. Cl. C07K 16/28; A61K 31/496; A61K 31/706; A61K 39/395

(52) U.S. Cl. CPC C07K 16/2866; A61K 31/496; A61K 31/706; A61K 39/3955; C07K 2317/35; C07K 2317/40; C07K 2317/732

(57) ABSTRACT

The present invention relates to anti-CD-25 antibodies for use in the treatment of acute myeloid leukemia (AML) and diffuse large B-cell lymphoma (DLBCL).

### FIELD OF THE INVENTION

The present invention is directed to anti-CD25 antibodies for use in the treatment of acute myeloid leukemia (AML) and diffuse large B-cell lymphoma (DLBCL).

### BACKGROUND TO THE INVENTION

CD25, also known, as the a-subunit of the interleukin-2 receptor (IL2RA) is a surface antigen that allows binding of IL-2 with a high affinity and subsequent signalling cascade. CD25 is constitutively expressed on regulatory T cells (Tregs) that rely on IL-2 consumption for their growth, and transiently upregulated on recently activated T cells. IL-2 is a key cytokine that plays an important role in the clonal expansion of antigen-specific T cells and the acquisition of their effector functions. The abundance of intratumoral Tregs, and in particular the ratio of Tregs to effector T cells (Teff) has been shown to be predictive of clinical outcome in a number of solid tumors in humans (Nishikawa and Sakaguchi, 2014, Curr Opin Immunol 27, 1-7; Wing et al., 2019, Immunity 50, 302-316). Indeed, Tregs contribute to an immune suppressive tumor microenvironment and several strategies to deplete them have been evaluated.

CD25 Mab (RG6292) is an afucosylated IgG1 antibody, non-IL-2 blocking that is shown to efficiently deplete Tregs in human tumor explants and preclinical mouse models of cancer while allowing a redistribution of IL-2 to Teff and the formation of anti-tumor adaptive immune responses (Solomon et al., 2020, Nature Cancer, 1 (12): 1153-1166). CD25 Mab binds to CD25+ target cells and its crystallisable fragment (Fc) to Fc receptors expressed on the surface of effector cells, such as Natural Killer (NK) cells, monocytes and macrophages. CD25 Mab mediates killing of target cells through antibody-dependent cellular cytotoxicity (ADCC) and antibody-dependent cellular phagocytosis (ADCP). CD25 Mab is currently being investigated in phase I monotherapy study as well as in phase Ib clinical trial in combination with Atezolizumab (anti PD-L1 antibody).

In addition to its role in healthy T cells, CD25 expression has also been described in a number of haematological malignancies (Flynn and Hartley, 2017, Br J Haematol 179, 20-35) such as T-and B-cell lymphomas as well as acute myeloid leukemia (AML). In particular, CD25 seems to be expressed on a subset of tumor cells in AML and diffuse large B-cell lymphoma (DLBCL) and its expression was associated with lower survival (Fujiwara et al., 2013, Hematology 18, 14-19; Gonen et al., 2012, Blood 120, 2297-2306).

Moreover, there is some evidence that CD25 may be restricted to leukemic stem cells or cells with a progenitor phenotype in AML (Aref et al., 2020, Leuk Res Rep 13, 100203; Kageyama Y et al., 2018, PLOS One 13(12) e.0209295). These cells are thought to play a role in the propagation of the disease and the high relapse rate, despite recent advances in the treatment of AML (Kantarjian et al., 2021, Blood Cancer J 11, 41). A higher frequency of Tregs was observed in the bone marrow of AML patients as compared to that of healthy volunteers and the abundance of Tregs correlated with poorer outcome in AML patients (Dong et al., 2020, Front Immunol 11, 1710). Thus, there is a need for further treatment options for AML and DLBCL.

Current treatment options for AML include anthracyclines-cytarabine regimens (such as a daunorubicin and cytarabine regime), FLT3 inhibitors (e.g. gilteritinib, midostaurin, Sorafenib), BCL-2 inhibitors (e.g. venetoclax), IDH inhibitors (e.g. enasidenib, ivosidenib), hypomethylating agents (e.g. azacitidine, decitabine) and antibody therapy (e.g. CD33 antibody gemtuzumab ozogamicin) and combination therapies thereof.

The present inventors have now found that anti-CD25 antibodies such as CD25 Mab (RG6292) potentially have a dual mode of action that depletes suppressive Tregs and has a direct cytotoxicity effect on the CD25+ malignant cells, providing antibodies that can be effective treatment for AML and DLBLC.

### SUMMARY OF THE INVENTION

The present invention provides anti-CD25 antibodies for use in the treatment of acute myeloid leukemia (AML) and diffuse large B-cell lymphoma (DLBCL).

In a first aspect of the invention there is provided an anti-CD25 antibody for use in the treatment of acute myeloid leukemia or diffuse large B-cell lymphoma.

A second aspect of the invention provides an anti-CD25 antibody for use in the treatment of acute myeloid leukemia or diffuse large B-cell lymphoma, wherein the anti-CD25 antibody is administered alone or in combination with one or more further therapeutic agents. The anti-CD25 antibody and the further therapeutic agent are for separate, simultaneous or sequential administration.

A third aspect of the invention provides a combination of an anti-CD25 antibody and a further therapeutic agent for use in the treatment of acute myeloid leukemia or diffuse large B-cell lymphoma, wherein the anti-CD25 antibody and the further therapeutic agent are for separate, simultaneous or sequential administration.

A fourth aspect of the invention provides a method of treating acute myeloid leukemia or diffuse large B-cell lymphoma in a subject comprising administrating to the subject an effective amount of an anti-CD25 antibody.

A fifth aspect of the invention provides the use of anti-CD25 antibody in the manufacture of a medicament for the treatment of acute myeloid leukemia or diffuse large B-cell lymphoma.

A sixth aspect of the invention provides the use of anti-CD25 antibody and a further therapeutic agent in the manufacture of a medicament for the treatment of acute myeloid leukemia or diffuse large B-cell lymphoma.

A seventh aspect of the invention provides a method of selecting a patient having acute myeloid leukemia for treatment with an anti-CD25 antibody, the method comprising: determining the expression level of CD25 on target cells in a sample from the patient, wherein if the cells have an expression level above about 900 CD25 molecules per cell the patient is suitable for treatment with the antibody.

An eighth aspect of the invention provides a method of selecting a patient having AML for treatment with an anti-CD25 antibody, the method comprising determining the presence or absence of an FLT3-ITD mutation in a sample from the patient, wherein if the mutation is present in the sample the patient is suitable for treatment with the antibody.

A ninth aspect of the invention provides a method of predicting the response of an AML patient to treatment with an anti-CD25 antibody, the method comprising determining the presence or absence of an FLT3-ITD mutation in a sample from the patient, wherein the presence of the mutation in the sample is indicative for a patient who will respond to treatment with the anti-CD25 antibody.

A tenth aspect of the invention provides a method of treating AML in a subject comprising administrating to the subject an effective amount of an anti-CD25 antibody, wherein the subject comprises the presence of an FLT3-ITD mutation.

An eleventh aspect of the invention provides a method for preventing or reducing the risk of relapse in an AML patient, the method comprising administering an anti-CD25 antibody to the patient.

A twelfth aspect of the invention provides a method for method for treating AML in a patient who has undergone BCL-2 inhibitor-hypomethylating agent combination treatment, the method comprising administering an anti-CD25 antibody to the patient.

### BRIEF DESCRIPTION OF FIGURES

FIG. 1—Shows the CD25 expression on target cells. The density of CD25 molecules on the surface of four target cells (iTregs, Pfeiffer, EOL-1 and AML-22) was determined using BD quantibrite™ Beads after 17h of incubation in ADCC assay medium. The CD25 density of iTregs and Pfeiffer cells is shown on the left Y-axis and that of EOL-1 and AML-22 on the right Y-axis. The limit of detection (L.O.D) represents the lowest number of PE molecules present on the BD quantibrite™ Beads and therefore the linear relationship between the number of PE molecules/cell and the median fluorescence intensity (MFI) is not guaranteed below this value.

FIG. 2—Shows the ADCC potential and CD25 density on Target cells induced by CD25

Mab treatment. (A) shows the ADCC activity of the tested compounds (CD25 Mab or isotype control antibodies) as a frequency of killing. The calculations are based on the target cell event count normalized to the number of target cells in the absence of effector NK cells and compounds. Flow cytometric analysis was performed 17 h after onset of ADCC assay. (B) shows the density of CD25 molecules on the surface of remaining live target cells (EOL-1 and AML-22) determined using BD quantibrite™ Beads, 17 h after ADCC assay initiation. The limit of detection (L.O.D) represents the lowest number of PE molecules present on the BD quantibrite™ Beads and therefore the linear relationship between the number of PE molecules/cell and the median fluorescence intensity (MFI) is not guaranteed below this value.

FIG. 3—Shows CD16 expression on NK cells. (A) shows the proportion of NK cells expressing CD16 after 17 h of co-incubation with EOL-1 or AML-22 cells and CD25 Mab or isotype control antibody. (B) shows the proportion of NK cells expressing CD16 after 17 h of co-incubation with Pfeiffer cells or iTregs and CD25 Mab or isotype control antibody.

FIG. 4—Shows the CD69 expression on NK cells. (A) shows the proportion of NK cells expressing CD69 after 17 h of co-incubation with EOL-1 or AML-22 cells and CD25 Mab or isotype control antibody. (B) shows the proportion of NK cells expressing CD69 after 17 h of co-incubation with Pfeiffer cells or iTregs and CD25 Mab or isotype control antibody.

FIG. 5—Shows CD25 expression on NK cells. (A) shows the proportion of NK cells expressing CD25 after 17 h of co-incubation with EOL-1 or AML-22 cells and CD25 Mab or isotype control antibody. (B) shows the proportion of NK cells expressing CD25 after 17 h of co-incubation with Pfeiffer cells or iTregs and CD25 Mab or isotype control antibody.

FIG. 6—Shows the correlation of ADCC activity with CD25 density. (A) shows the ADCC activity of the tested compounds (CD25 Mab or isotype control antibodies) as a frequency of the relative luminescence units normalized to the maximum signal obtained for each cell line and NK cell donor pair. The results were obtained from two independent experiments, with NK cells isolated from a total of four blood donors. Cytotoxicity evaluation was performed via CytoTox-Glo™ luminescence readout 16-20 hours after ADCC assay onset. (B) shows the EC50 values derived from ADCC assays with a titration of CD25 Mab using CytoTox-Glo™ or flow cytometry as readouts (three independent experiment with two NK cell donors in each). EC50 values were calculated using Prism 8 (GraphPad software) and its inbuilt non-linear regression curve fit (log (agonist) vs response, variable slope, 4 parameter)). (C) shows the density of CD25 molecules on the surface of live target cells (Pfeiffer, EOL-1 and AML-22) determined using BD quantibrite™ Beads, 16-20 h after ADCC assay initiation. The limit of detection (L.O.D) represents the lowest number of PE molecules present on the BD quantibrite™ Beads and therefore the linear relationship between the number of PE molecules/cell and the median fluorescence intensity (MFI) is not guaranteed below this value. The results were obtained from three independent experiments.

FIG. 7—Shows killing activity of CD25+ AML cells and of Treg (A) Killing activity of the tested compounds at 10 μg/ml (CD25 Mab or isotype control antibodies) as a frequency of CD25+ AML cell killing. Positive control EOL-1 cell line and four AML patient samples were used as target cells. Flow cytometric analysis was performed 20 h after onset of ADCC assay. Mean +/−SEM represent results obtained with two NK cell donors and technical duplicates. Multiple t tests, Holm-Sidak correction for multiple comparisons. (B): Treg killing activity as described above for (A). Mean +/−SEM represent results obtained with AML Treg (n=2) or healthy BM Treg (n=2). Multiple t tests, Holm-Sidak correction for multiple comparisons. Significance levels are indicated as follows: ns, not significant=P>0.05; *, P≤0.05; **, P≤0.01; ***, P≤0.001; ****, P≤0.0001.

### DETAILED DESCRIPTION OF THE INVENTION

The present invention provides anti-CD25 antibodies for use in the treatment of acute myeloid leukemia (AML) or diffuse large B-cell lymphoma (DLBCL) in a subject, and methods of treating acute myeloid leukemia or diffuse large B-cell lymphoma in a subject using anti-CD25 antibodies.

The inventors have found that anti-CD25 antibodies were able to deplete CD25+ malignant cells with a wide range of CD25 expression levels. The inventors have shown the direct killing of CD25+ AML and DLBCL cells with the anti-CD25 antibody, in particular CD25+ AML blast cells. In particular the inventors have found that despite the low level of expression of CD25 on tumor cells associated with AML anti-CD25 antibodies are effective in killing these tumor cells. The inventors have found anti-CD25 antibodies can be used for targeting cancer cells having low CD25 expression, and therefore are also suitable for use in the treatment of cancers such as AML where there can be low level of CD25 expression on the blast cells.

CD25 is the alpha chain of the IL-2 receptor, and is found on activated T cells, regulatory T cells, activated B cells, some NK T cells, some thymocytes, myeloid precursors and oligodendrocytes. Low CD25 expression may also be found on blast cells of AML patients. CD25 associates with CD122 and CD132 to form a heterotrimeric complex that acts as the high-affinity receptor for IL-2. The consensus sequence of human CD25 is shown below and identified as SEQ ID NO:1 (Uniprot accession number P01589; the extracellular domain of mature human CD25, corresponding to amino acids 22-240 is underlined).

```
                10 20 30 40
MDSYLLMWGL LTFIMVPGCQ AELCDDDPPE IPHATFKAMA
                50 60 70 80
YKEGTMLNCE CKRGFRRIKS GSLYMLCTGN SSHSSWDNQC
                90 100 110 120
QCTSSATRNT TKQVTPQPEE QKERKTTEMQ SPMQPVDQAS
              130 140 150 160
LPGHCREPPP WENEATERIY HFVVGQMVYY QCVQGYRALH
              170 180 190 200
RGPAESVCKM THGKTRWTQP QLICTGEMET SQFPGEEKPQ
              210 220 230 240
ASPEGRPESE TSCLVTTTDF QIQTEMAATM ETSIFTTEYQ
              250 260 270
VAVAGCVELL ISVLLLSGLT WQRRQRKSRR TI
```

As used herein an “anti-CD25 antibody” or an “an antibody that binds CD25” refers to an antibody that is capable of binding to the CD25 subunit of the IL-2 receptor. This subunit is also known as the alpha subunit of the IL-2 receptor.

An anti-CD25 antibody is an antibody capable of specific binding to the CD25 subunit (antigen) of the IL-2 receptor. “Specific binding”, “bind specifically”, and “specifically bind” are understood to mean that the antibody has a dissociation constant (Kd) for the antigen of interest of less than about 10⁻⁶ M, 10⁻⁷ M, 10⁻⁸ M, 10⁻⁹ M, 10⁻¹⁰ M, 10⁻¹¹ M, 10⁻¹² M or 10⁻¹³ M. In a preferred embodiment, the dissociation constant is less than 10⁻⁸ M, for instance in the range of 10⁻⁹ M, 10⁻¹⁰ M, 10⁻¹¹ M, 10⁻¹² M or 10⁻¹³ M.

Anti-CD25 antibodies suitable for use in the present invention include for example those described in WO2017/174331, WO2018/167104, WO2019/008386, WO2019/175215, WO2019/175216, WO2019/175217, WO2019/175220, WO2019/17522. WO2019/175223, WO2019/175224, WO2019/175226, the contents which are incorporated herein by reference.

As used herein, the term “antibody” refers to both intact immunoglobulin molecules as well as fragments thereof that include the antigen-binding site, and includes polyclonal, monoclonal, genetically engineered and otherwise modified forms of antibodies, including but not limited to chimeric antibodies, humanised antibodies, heteroconjugate and/or multispecific antibodies (e.g., bispecific antibodies, diabodies, tribodies, and tetrabodies), and antigen binding fragments of antibodies, including e.g. Fab′, F(ab′)₂, Fab, Fv, rIgG, polypeptide-Fc fusions, single chain variants (scFv fragments, VHHs, Trans-bodies®, Affibodies®, shark single domain antibodies, single chain or Tandem diabodies (TandAb®), VHHs, Anticalins®, Nanobodies®, minibodies, BiTE®s, bicyclic peptides and other alternative immunoglobulin protein scaffolds). In some embodiments, an antibody may lack a covalent modification (e.g., attachment of a glycan) that it would have if produced naturally. In some embodiments, an antibody may contain a covalent modification (e.g., attachment of a glycan, a detectable moiety, a therapeutic moiety, a catalytic moiety, or other chemical group providing improved stability or administration of the antibody, such as poly-ethylene glycol). In some embodiments, the antibody may be in the form of a masked antibody (e.g. Probodies®). A masked antibody can comprise a blocking or “mask” peptide that specifically binds to the antigen binding surface of the antibody and interferes with the antibody's antigen binding. The mask peptide is linked to the antibody by a cleavable linker (e.g. by a protease). Selective cleavage of the linker in the desired environment, i.e. in the tumour environment, allows the masking/blocking peptide to dissociate, enabling antigen binding to occur in the tumour, and thereby limiting potential toxicity issues. “Antibody” may also refer to camelid antibodies (heavy-chain only antibodies) and antibody-like molecules such as anticalins (Skerra (2008) FEBS J 275, 2677-83). In some embodiments, an antibody is polyclonal or oligoclonal, that is generated as a panel of antibodies, each associated to a single antibody sequence and binding more or less distinct epitopes within an antigen (such as different epitopes within human CD25 extracellular domain that are associated to different reference anti-human CD25 antibodies). Polyclonal or oligoclonal antibodies can be provided in a single preparation for medical uses as described in the literature (Kearns J D et al., 2015. Mol Cancer Ther. 14:1625-36).

The antibodies used in the present invention may be monospecific, bispecific, or multispecific. “Multispecific antibodies” may be specific for different epitopes of one target antigen or polypeptide, or may contain antigen-binding domains specific for more than one target antigen or polypeptide. In some embodiments of the invention the antibody is monospecific. In some embodiments the antibody binds CD25 in a monovalent manner (i.e. a ratio of one antibody to one CD25 molecule). In further embodiments the antibody is a monospecific bivalent antibody, i.e. the antibody binds CD25 in a ratio of one antibody to two CD25 molecules.

In some embodiments of the invention the antibody is monoclonal. The antibody may additionally or alternatively be humanised or human. In a further embodiment, the antibody is human, or in any case an antibody that has a format and features allowing its use and administration in human subjects.

As used herein, “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced.

As used herein, “human antibody” refers to antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Antibodies (Abs) and immunoglobulins (Igs) are glycoproteins having the same structural characteristics. Immunoglobulins may be from any class such as IgA, IgD, IgG, IgE or IgM. Immunoglobulins can be of any subclass such as IgG1, IgG2, IgG3, or IgG4. In a preferred embodiment of the invention the anti-CD25 antibody is from the IgG class, preferably the IgG1 subclass. In one embodiment, the anti-CD25 antibody is from the human IgG1 subclass.

In a preferred embodiment of the invention, the anti-CD25 antibody binds FcγR with high affinity, preferably an activating receptor with high affinity. Preferably the antibody binds FcγRI and/or FcγRIIa and/or FcγRIIIa with high affinity. In a particular embodiment, the antibody binds to at least one activatory Fcγreceptor with a dissociation constant of less than about 10⁻⁶M, 10⁻⁷M, 10⁻⁸M, 10⁻⁹M or 10⁻¹⁰M.

In some embodiments, the antibody is an IgG1 antibody, preferably a human IgG1 antibody, which is capable of binding to at least one Fc activating receptor. For example, the antibody may bind to one or more receptor selected from FcγRI, FcγRIIa, FcγRIIc, FcγRIIIa and FcγRIIIb. In some embodiments, the antibody is capable of binding to FcγRIIIa. In some embodiments, the antibody is capable of binding to FcγRIIIa and FcγRIIa and optionally FcγRI. In some embodiments, the antibody is capable of binding to these receptors with high affinity, for example with a dissociation constant of less than about 10⁻⁷M, 10⁻⁸M, 10⁻⁹M or 10⁻¹⁰M.

In some embodiments, the antibody binds an inhibitory receptor, FcγRIIb, with low affinity. In some embodiments, the antibody binds FcγRIIb with a dissociation constant higher than about 10⁻⁷ M, higher than about 10⁻⁶ M or higher than about 10⁻⁵M.

In some embodiments the antibody may be afucosylated. The Fc region of the antibody can be modified to change the glycosylation profile using known techniques in the art. Available techniques to produce antibodies with absent or reduce fucosylation profiles, include commercially available technology such as GlyMAXX (ProBiogen) and methods such as those disclosed in WO2011/035884.

In some embodiments the anti-CD25 antibody induces ADCC activity. The anti-CD25antibody exhibits ADCC activity against CD25+ target cells. “Antibody-dependent cell-mediated cytotoxicity” (ADCC) refers to a cell-mediated reaction in which nonspecific cytotoxic cells that express Fc receptors (FcRs) (e.g. Natural Killer (NK) cells, neutrophils, and macrophages) recognize bound antibody on a target cell and thereby lead to lysis of the target cell. In some embodiments the anti-CD25 antibody induces ADCP activity. “Antibody-dependent cell-mediated phagocytosis” (ADCP) refers to a cell-mediated reaction in which phagocytes (such as macrophages) that express Fc receptors (FcRs) recognize bound antibody on a target cell and thereby lead to phagocytosis of the target cell.

The anti-CD25 antibody used in the invention may function through ADCC and ADCP activity. ADCC and ADCP can be measured using assays that are known and available in the art.

In some embodiments of the invention the anti-CD25 antibody does not inhibit the binding of Interleukin-2 to CD25. References herein to “does not inhibit the binding of Interleukin-2 to CD25” may alternatively be expressed as the anti-CD25 antibody is a non-IL-2 blocking antibody or a “non-blocking” antibody (with respect to the non-blocking of IL-2 binding to CD25 in the presence of the anti-CD25 antibody), i.e. the antibody does not block the binding of Interleukin-2 to CD25 and in particular does not inhibit Interleukin-2 signalling in CD25-expressing cells. References herein to a non-IL-2 blocking antibody may alternatively be expressed as an anti-CD25 antibody that “does not inhibit the binding of Interleukin-2 to CD25” or as an anti-CD25 antibody that “does not inhibit the signalling of IL-2”. References to “non-blocking” “non-IL2 blocking”, “does not block”, or “without blocking” and the like (with respect to the non-blocking of IL-2 binding to CD25 in the presence of the anti-CD25 antibody) include embodiments wherein the anti-CD25 antibody of the invention does not block the signalling of IL-2 via CD25. That is the anti-CD25 antibody inhibits less than 50% of IL-2 signalling compared to IL-2 signalling in the absence of the antibodies. In particular embodiments of the invention as described herein, the anti-CD25 antibody inhibits less than about 50%, 40%, 35%, 30%, preferably less than about 25% of IL-2 signalling compared to IL-2 signalling in the absence of the antibodies.

Some anti-CD25 antibodies may allow binding of IL-2 to CD25, but still block signalling via the CD25 receptor. The non-IL-2 blocking anti-CD25 antibodies allow binding of IL-2 to CD25 to facilitate at least 50% of the level of signalling via the CD25 receptor compared to the signalling in the absence of the anti-CD25 antibody.

IL-2 signalling via CD25 may be measured by methods as discussed for example in WO2018/167104 and as known in the art. Comparison of IL-2 signalling in the presence and absence of the anti-CD25 antibody agent can occur under the same or substantially the same conditions.

In some embodiments, IL-2 signalling can be determined by measuring by the levels of phosphorylated STAT5 protein in cells, using a standard Stat-5 phosphorylation assay. For example, a Stat-5 phosphorylation assay to measure IL-2 signalling may involve culturing PMBC cells in the presence of the anti-CD25 antibody at a concentration of 10 ug/ml for 30 mins and then adding varying concentrations of IL-2 (for example 10 U/ml or vary concentrations of 0.25 U/ml, 0.74 U/ml, 2.22 U/ml, 6.66 U/ml or 20 U/ml) for 10 mins. Cells may then be permeabilized and levels of STAT5 protein can then be measured with a fluorescent labelled antibody to a phosphorylated STAT5 peptide analysed by flow cytometry. The percentage blocking of IL-2 signalling can be calculated as follows: % blocking=100×[(% Stat5+ cells No Antibody group−% Stat5+ cells 10 ug/ml Antibody group)/(% Stat5+ cells No Ab group).

Examples of non-blocking anti-CD25 antibodies are described in WO2018/167104, WO2019/175215, WO2019/175216, WO2019/175217, WO2019/175220, WO2019/17522. WO2019/175223, WO2019/17524, WO2019/17526 the contents of which are incorporated herein by reference in their entirety.

The anti-CD25 antibody may specifically bind to an epitope within the extracellular region of human CD25. In some embodiments the antibody binds to an epitope that is distinct from the IL-2 binding site and and does not block the binding of IL-2 to CD25.

As used herein, “epitope” refers to a portion of an antigen that is bound by an antibody or antigen-binding fragment. As is well known in the art, epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents.

An epitope is conformational in that it is comprised of portions of an antigen that are not covalently contiguous in the antigen but that are near to one another in three-dimensional space when the antigen is in a relevant conformation. For example, for CD25, conformational epitopes are those comprised of amino acid residues that are not contiguous in CD25 extracellular domain; linear epitopes are those comprised of amino acid residues that are contiguous in CD25 extracellular domain. Means for determining the exact sequence and/or particularly amino acid residues of the epitope for the anti-CD25 antibody are known in the literature, including competition with peptides, from antigen sequences, binding to CD25 sequence from different species, truncated, and/or mutagenized (e.g. by alanine scanning or other site-directed mutagenesis), phage display-based screening, yeast presentation technologies, or (co-) crystallography techniques. Methods of determining spatial conformation of epitopes are also well known in the art and include, for example, x-ray crystallography and 2-D nuclear magnetic resonance. See, for example, Epitope Mapping Protocols in Methods in Molecular Biology, Vol. 66, Glenn E. Morris, Ed (1996). Therefore, in some embodiments the anti-CD25 antibody may recognise a conformational epitope.

In some embodiments the anti-CD25 antibody binds to an epitope wherein the epitope comprises one or more amino acid residues comprised in one or more of the amino acid stretches selected from amino acids 150-163 of SEQ ID NO:1 (YQCVQGYRALHRGP) (SEQ ID NO: 52), amino acids 166-186 of SEQ ID NO:1 (SVCKMTHGKTRWTQPQLICTG) (SEQ ID NO: 53), amino acids 42-56 of SEQ ID NO:1 (KEGTMLNCECKRGFR) (SEQ ID NO: 54) and amino acids 70-88 of SEQ ID NO:1 (NSSHSSWDNQCQCTSSATR) (SEQ ID NO: 55). Preferably the epitope comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen or more amino acid residues comprised in one of more the amino acid stretches selected from amino acids 150-163 of SEQ ID NO:1 (YQCVQGYRALHRGP) (SEQ ID NO: 52), amino acids 166-186 of SEQ ID NO:1 (SVCKMTHGKTRWTQPQLICTG) (SEQ ID NO: 53), amino acids 42-56 of SEQ ID NO:1 (KEGTMLNCECKRGFR) (SEQ ID NO: 54) and/or amino acids 70-88 of SEQ ID NO:1 (NSSHSSWDNQCQCTSSATR) (SEQ ID NO: 55).

In some embodiments the anti-CD25 antibody binds to an epitope of human CD25 wherein the epitope comprises at least one sequence selected from amino acids 150-158 of SEQ ID NO:1 (YQCVQGYRA) (SEQ ID NO: 56), amino acids 176-180 of SEQ ID NO:1 (RWTQP) (SEQ ID NO: 57), amino acids 42-56 of SEQ ID NO:1 (KEGTMLNCECKRGFR) (SEQ ID NO: 54) and amino acids 74-84 of SEQ ID NO:CD25.

In a one embodiment the anti-CD25 antibody binds to an epitope comprising the sequence of amino acids 70-84 of SEQ ID NO:1 (NSSHSSWDNQCQCTS) (SEQ ID NO: 59).

Native antibodies and immunoglobulins are usually heterotetrameric glycoproteins of about 150,000 daltons, composed of two identical light (L) chains and two identical heavy (H) chains. Each heavy chain has at the amino terminus a variable domain (V_{H}) followed by a number of constant domains. Each light chain has a variable domain at the amino terminus V_{L}) and a constant domain at the carboxy terminus.

The variable regions are capable of interacting with a structurally complementary antigenic target and are characterized by differences in amino acid sequence from antibodies of different antigenic specificity. The variable regions of either heavy or light chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions.

These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed as H1, H2, H3, L1, L2, L3) for the respective heavy (H) and light (L) chains. The CDR regions specified herein are defined according to Kabat (Kabat et al., 1977. J Biol Chem 252, 6609-6616).

In some embodiments the anti-CD25 antibody is selected from the group consisting of:

(a) an antibody or antigen binding fragment thereof comprising:
a heavy chain variable region comprising a CDR-H1 comprising the amino acid sequence of any one of SEQ ID NOs: 2-5, a CDR-H2 comprising the amino acid sequence of any one of SEQ ID NOs: 6-11 and a CDR-H3 comprising the amino acid sequence of SEQ ID NO: 12, and
a light chain variable region comprising a CDR-L1 comprising the amino acid sequence of SEQ ID NO: 13, CDR-L2 comprising the amino acid sequence of SEQ ID NO: 14, and a CDR-L3 comprising the amino acid sequence of SEQ ID NO: 15;
   (b) an antibody or antigen binding fragment thereof comprising:
a heavy chain variable region comprising a CDR-H1 comprising the amino acid sequence of SEQ ID NO: 23, a CDR-H2 comprising the amino acid sequence of SEQ ID NO: 24, and a CDR-H3 comprising the amino acid sequence of SEQ ID NO:25, and a light chain variable region comprising a CDR-L1 comprising the amino acid sequence of SEQ ID NO: 26, a CDR-L2 comprising the amino acid sequence of SEQ ID NO 27, and a CDR-L3 comprising the amino acid sequence of SEQ ID NO:28; and
   (c) an antibody or antigen binding fragment thereof comprising:
a heavy chain variable region comprising a CDR-H1 comprising the amino acid sequence of any one of SEQ ID NOs: 31-33, a CDR-H2 comprising the amino acid sequence of any one of SEQ ID NOs: 34-38, and a CDR-H3 comprising the amino acid sequence of SEQ ID NO: 39, and
a light chain variable region comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO:42.

In some embodiments the anti-CD25 antibody is selected from the group consisting of:

(a) an antibody or antigen binding fragment thereof comprising:
a heavy chain variable region comprising a CDR-H1 comprising the amino acid sequence of SEQ ID NO: 2, a CDR-H2 comprising the amino acid sequence of SEQ ID NO: 6 and a CDR-H3 comprising the amino acid sequence of SEQ ID NO: 12; and a light chain variable region comprising a CDR-L1 comprising the amino acid sequence of SEQ ID NO: 13, a CDR-L2 comprising the amino acid sequence of SEQ ID NO:14, and a CDR-L3 comprising the amino acid sequence of SEQ ID NO: 15;
   b) an antibody or antigen binding fragment thereof comprising:
a heavy chain variable region comprising a CDR-H1 comprising the amino acid sequence of SEQ ID NO: 2, a CDR-H2 comprising the amino acid sequence of SEQ ID NO: 7 and a CDR-H3 comprising the amino acid sequence of SEQ ID NO: 12; and
a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
   c) an antibody or antigen binding fragment thereof comprising:
a heavy chain variable region comprising a CDR-H1 comprising the amino acid sequence of SEQ ID NO: 3, a CDR-H2 comprising the amino acid sequence of SEQ ID NO: 8 and a CDR-H3 comprising the amino acid sequence of SEQ ID NO: 12; and a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
   d) an antibody or antigen binding fragment thereof comprising:
a heavy chain variable region comprising a CDR-H1 comprising the amino acid sequence of SEQ ID NO: 2, a CDR-H2 comprising the amino acid sequence of SEQ ID NO: 9 and a CDR-H3 comprising the amino acid sequence of SEQ ID NO: 12; and
a light chain variable region comprising a CDR-L1 comprising the amino acid sequence of SEQ ID NO: 13, a CDR-L2 comprising the amino acid sequence of SEQ ID NO:14, and a CDR-L3 comprising the amino acid sequence of SEQ ID NO: 15;
   e) an antibody or antigen binding fragment thereof comprising:
a heavy chain variable region comprising a CDR-H1 comprising the amino acid sequence of SEQ ID NO: 4, a CDR-H2 comprising the amino acid sequence of SEQ ID NO: 10 and a CDR-H3 comprising the amino acid sequence of SEQ ID NO: 12; and
a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15; and
   f) an antibody or antigen binding fragment thereof comprising:
a heavy chain variable region comprising a CDR-H1 comprising the amino acid sequence of SEQ ID NO: 5, a CDR-H2 comprising the amino acid sequence of SEQ ID NO: 11 and a CDR-H3 comprising the amino acid sequence of SEQ ID NO: 12; and
a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15.

In some embodiments the anti-CD25 antibody is selected from the group consisting of:
a) an antibody comprising a heavy chain variable region comprising the amino acid sequence of any one of SEQ ID NO: 16-21 and a light chain variable region comprising the amino acid sequence of SEQ ID NO: 22;
   b) an antibody comprising a heavy chain variable region comprising the amino acid sequence of SEQ ID NO: 29 and a light chain variable region comprising the amino acid sequence of SEQ ID NO:30; and
   c) an antibody comprising a heavy chain variable region comprising the amino acid sequence of any one of SEQ ID NO: 43-48 and a light chain variable region comprising the amino acid sequence of SEQ ID NO:49.

In some embodiments the anti-CD25 antibody is selected from the group consisting of:

a) an antibody comprising a heavy chain variable region comprising the amino acid sequence of SEQ ID NO: 16 and a light chain variable region comprising the amino acid sequence of SEQ ID NO:22;
b) an antibody comprising a heavy chain variable region comprising the amino acid sequence of SEQ ID NO: 17 and a light chain variable region comprising the amino acid sequence of SEQ ID NO: 22;
c) an antibody comprising a heavy chain variable region comprising the amino acid sequence of SEQ ID NO: 18 and a light chain variable region comprising the amino acid sequence of SEQ ID NO: 22;
d) an antibody comprising a heavy chain variable region comprising the amino acid sequence of SEQ ID NO: 19 and a light chain variable region comprising the amino acid sequence of SEQ ID NO: 22;
e) an antibody comprising a heavy chain variable region comprising the amino acid sequence of SEQ ID NO: 20 and a light chain variable region comprising the amino acid sequence of SEQ ID NO: 22;
f) an antibody comprising a heavy chain variable region comprising the amino acid sequence of SEQ ID NO: 21 and a light chain variable region comprising the amino acid sequence of SEQ ID NO: 22; and
g) an antibody comprising a heavy chain variable region comprising a sequence having at least 80%, at least 85%, at least 90%, at least 95%, at least 96%, at least 97%, at least 98% or at least 99% sequence identity to any one of SEQ ID NO: 16-21 and a light chain variable region comprising a sequence having at least 80%, at least 85%, at least 90%, at least 95%, at least 96%, at least 97%, at least 98% or at least 99% sequence identity to SEQ ID NO: 22.

The SEQ ID NOs for the complementarity determining regions (HCDR1-3 and LCDR1-3), and heavy and light chain variable region of exemplified antibodies are provided in the below table:

TABLE 1

| Antibody | HCDR1 | HCDR2 | HCDR2 | VH | LCDR1 | LCDR2 | LCDR3 | VL |
|---|---|---|---|---|---|---|---|---|
| aCD25-a-686 | 2 | 6 | 12 | 16 | 13 | 14 | 15 | 22 |
| aCD25-a-686-m1 | 2 | 7 | 12 | 17 | 13 | 14 | 15 | 22 |
| aCD25-a-686-m2 | 3 | 8 | 12 | 18 | 13 | 14 | 15 | 22 |
| aCD25-a-686-m3 | 2 | 9 | 12 | 19 | 13 | 14 | 15 | 22 |
| aCD25-a-686-m4 | 4 | 10 | 12 | 20 | 13 | 14 | 15 | 22 |
| aCD25-a-686-m5 | 5 | 11 | 12 | 21 | 13 | 14 | 15 | 22 |
| aCD25-a-674 | 23 | 24 | 25 | 29 | 26 | 27 | 28 | 30 |
| aCD25-a-646 | 31 | 34 | 39 | 43 | 40 | 41 | 42 | 49 |
| aCD25-a-646-m1 | 32 | 35 | 39 | 44 | 40 | 41 | 42 | 49 |
| aCD25-a-646-m2 | 33 | 36 | 39 | 45 | 40 | 41 | 42 | 49 |
| aCD25-a-646-m3 | 33 | 37 | 39 | 46 | 40 | 41 | 42 | 49 |
| aCD25-a-646-m4 | 33 | 35 | 39 | 47 | 40 | 41 | 42 | 49 |
| aCD25-a-646-m5 | 33 | 38 | 39 | 48 | 40 | 41 | 42 | 49 |

Such antibodies are further described in WO2019/175216, WO2019/175217 and WO2019/1175222. The contents which is incorporated herein by reference.

The antibody referred to herein as aCD25-a-686 may also be referred to as RG6292. In a preferred embodiment the anti-CD25 antibody is RG6292. The anti-CD25 antibody referred to as “RG6292”, is an afucosylated human IgG1 monoclonal antibody. RG6292 has a heavy chain sequence having the sequence of SEQ ID NO: 50 and a light chain sequence having the sequence of SEQ ID NO: 51.

Such antibodies are known to be “non-IL-2 blocking” antibodies and do not inhibit the binding of IL-2 to CD25.

Variants of the above defined antibodies can also be used. Variants of the antibodies include antibodies wherein the sequence for each CDR sequence comprises an amino acid sequence with:
(i) at least 85% identity thereto, and/or
(ii) one, two, or three amino acid substitutions relative to SEQ ID NOs: 2-15, 23-28, or 31-42.

Variants of the antibodies also include antibodies wherein the sequence for each of the light chain and heavy chains comprise an amino acid sequence with:
(i) at least 80% identity thereto, and/or
(ii) one, two, three, four or five amino acid substitutions relative to SEQ ID NOs: 16-22, 29-30 or 43-51.

For example, one embodiment of the invention provides an anti-CD25 antibody for use in the treatment of AML selected from the group comprising:
a) antibody or antigen binding fragment thereof comprising:
   a heavy chain variable region comprising a CDR-H1 comprising the amino acid sequence having at least 85% sequence identity to any one of SEQ ID NOs: 2-5, a CDR-H2 comprising the amino acid sequence having at least 85% sequence identity to any one of SEQ ID NOs: 6-11 and a CDR-H3 comprising the amino acid sequence having at least 85% sequence identity to SEQ ID NO: 12, and
   a light chain variable region comprising a CDR-L1 comprising the amino acid sequence having at least 85% sequence identity to SEQ ID NO: 13, CDR-L2 comprising the amino acid sequence having at least 85% sequence identity to SEQ ID NO: 14, and a CDR-L3 comprising the amino acid sequence having at least 85% sequence identity to SEQ ID NO: 15;
b) antibody or antigen binding fragment thereof comprising:
   a heavy chain variable region comprising a CDR-H1 comprising the amino acid sequence having one, two, or three, amino acid substitutions relative to any one of SEQ ID NOs: 2-5, a CDR-H2 comprising the amino acid sequence having one, two, or three amino acid substitutions relative to any one of SEQ ID NOs: 6-11 and a CDR-H3 comprising the amino acid sequence having one, two, or three, amino acid substitutions relative to SEQ ID NO: 12, and
   a light chain variable region comprising a CDR-L1 comprising the amino acid sequence having one, two, or three, amino acid substitutions relative to SEQ ID NO: 13, CDR-L2 comprising the amino acid sequence having one, two, or three, amino acid substitutions relative to SEQ ID NO:14, and a CDR-L3 comprising the amino acid sequence having one, two, or three, amino acid substitutions relative to SEQ ID NO: 15; and
c) antibody or antigen binding fragment thereof comprising:
   a heavy chain variable region comprising:
      i) an amino acid sequence having at least 80% sequence identity to any one of SEQ ID NOs: 16-21; or
      ii) an amino acid sequence having one, two, three, four or five amino acid substitutions compared to SEQ ID NOs: 16-21;
   and
   a light chain variable region comprising:
      i) an amino acid sequence having at least 80% sequence identity to SEQ ID NO: 22; or
      ii) an amino acid sequence having one, two, three, four or five amino acid substitutions compared to SEQ ID NO: 22.

Percent (%) identity as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptides or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)). The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity =number of identical positions/total number of positions×100). Generally, references to % identity herein refer to % identity along the entire length of the molecule, unless the context specifies or implies otherwise.

In some embodiments the anti-CD25 antibody kills cancer, Treg, AML blast and/or PMBC cells. In some embodiments the antibody kills Treg cells and blast cells having an CD25 expression level of above about 900 CD25 molecules per cell. Preferably the antibody kills Treg cells and blast cells having an CD25 expression level of above about 1000 CD25 molecules per cell, preferably in the range of from about 1000 to 40000, from about 1000 to about 5000 or from about 1000 to about 2500 CD25 molecules per cell.

The expression level of CD25 on specific cells may also be referred to the CD25 density and is a measure of the number of CD25 molecules per cell. The expression level or density of CD25 on a cell can be determined as discussed in the Example and as known in the art, for example by flow cytometry. Cells having a CD25 of about 1000 CD25 molecules per cells are considered low expressing CD25 cells, in comparison to cells such as Treg cells which have high CD25 expression. Such low expressing CD25 cells include AML blasts.

In some embodiments of the invention, the anti-CD25 antibodies are for use in killing low expressing CD25 cells, such as CD25+ AML blast cells.

In some embodiments the anti-CD25 antibody induces a maximum reduction in CD16 expression on NK cells of 25%, when the antibody and NK cells are co-incubated with cells expressing 900 to 5000 CD25 molecules per cells. Preferably the NK cells are CD56dim NK cells. Reduction of CD16 expression can be measured by methods as discussed for example in the Example and known in the art.

The invention relates to the treatment of acute myeloid leukemia (AML) or diffuse large B-cell lymphoma (DLBCL). Preferably the invention relates to the treatment of acute myeloid leukemia (AML). Acute myeloid leukemia (AML) is a haematological cancer in which there is growth of abnormal cells of the myeloid lineage populations such as myeloblasts, red blood cells, and platelets which proliferate and accumulate in the bone marrow and spread to the blood. Classification schemes for AML are known in the art, e.g. the WHO 2008 classification of AML and the French-American-British (FAB) classification. Diffuse large B-cell lymphoma (DLBCL) is an aggressive type of non-Hodgkin lymphoma.

The anti-CD25 antibodies can be used to target the AML blast cells of the patient. In some embodiments the CD25 expression level on tumor cells from the subject being treated is at least about 900 CD25 molecules per cell. In some embodiments the CD25 expression level on tumor cells from the subject being treated is in the range from about 900 to about 5000 CD25 molecules per cell.

Reference to “treatment”, “treat” or “treating” of AML or DLBCL as used herein refers to any administration of a substance (e.g., the anti-CD25 antibody) that partially or completely alleviates, ameliorates, relives, inhibits, delays onset of, reduces severity of, and/or reduces incidence of one or more symptoms. A positive therapeutic effect may be for example, a reduced number of cancer cells, i.e. a reduction in AML blast cells.

The subject of any of the embodiments of the invention as described herein, is preferably mammal, preferably a cat, dog, horse, donkey, sheep, pig, goat, cow, hamster, mouse, rat, rabbit or guinea pig, but most preferably the subject is a human. Thus, in all aspects of the invention as described herein the subject is preferably a human. The subject may also be referred to herein as the patient.

The dosage regimen of a therapy described herein that is effective to treat a cancer patient may vary according to factors such as the disease state, age, and weight of the patient, and the ability of the therapy to elicit an anti-cancer response in the subject. The anti-CD25 antibody may be used in a therapeutically effective amount. As used herein, the term “therapeutically effective amount” means an amount (e.g., of an agent or of a pharmaceutical composition) that is sufficient, when administered to a population suffering from or susceptible to a disease and/or condition in accordance with a therapeutic dosing regimen, to treat such disease and/or condition. A therapeutically effective amount is one that reduces the incidence and/or severity of, stabilizes, and/or delays onset of, one or more symptoms of the disease, disorder, and/or condition. Those of ordinary skill in the art will appreciate that a “therapeutically effective amount” does not in fact require successful treatment to be achieved in a particular subject.

Selection of an appropriate dosage will be within the capability of one skilled in the art. For example 0.01, 0.1, 0.3, 0.5, 1, 2, 3, 4, 5, 6, 7, 8, 9, 10, 20, 30, 40, or 50 mg/kg. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). The dosage may also be varied for route of administration, the cycle of treatment, or consequently to dose escalation protocol that can be used to determine the maximum tolerated dose and dose limiting toxicity (if any) in connection to the administration of the antibody at increasing doses.

In some embodiments, a dosing regimen comprises a plurality of doses each of which are separated from one another by a time period of the same length. Alternatively, a dosing regimen comprises a plurality of doses and at least two different time periods separating individual doses. In some embodiments, all doses within a dosing regimen are of the same unit dose amount. Alternatively, different doses within a dosing regimen are of different amounts. In some embodiments, a dosing regimen comprises a first dose in a first dose amount, followed by one or more additional doses in a second dose amount different from the first dose amount. A dosing regimen may comprise a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The anti-CD25 antibody according to any aspect of the invention as described herein may be in the form of a pharmaceutical composition which additionally comprises a pharmaceutically acceptable carrier, diluent or excipient. These compositions include, for example, liquid, semi-solid and solid dosage formulations, such as liquid solutions (e.g., injectable and infusible solutions), dispersions or suspensions, tablets, pills, or liposomes. In some embodiments, a preferred form may depend on the intended mode of administration and/or therapeutic application. Pharmaceutical compositions containing the antibody can be administered by any appropriate method known in the art, including, without limitation, oral, mucosal, by-inhalation, topical, buccal, nasal, rectal, or parenteral (e.g. intravenous, infusion, intratumoural, intranodal, subcutaneous, intraperitoneal, intramuscular, intradermal, transdermal, or other kinds of administration involving physical breaching of a tissue of a subject and administration of the pharmaceutical composition through the breach in the tissue). Such a formulation may, for example, be in a form of an injectable or infusible solution that is suitable for intradermal, intratumoural or subcutaneous administration, or for intravenous infusion. The administration may involve intermittent dosing. Alternatively, administration may involve continuous dosing (e.g., perfusion) for at least a selected period of time, simultaneously or between the administration of other compounds. In some embodiments, the antibody can be prepared with carriers that protect it against rapid release and/or degradation, such as a controlled release formulation, such as implants, transdermal patches, and microencapsulated delivery systems. Biodegradable, biocompatible polymers can be used.

Those skilled in the art will appreciate, for example, that route of delivery (e.g., oral vs intravenous vs subcutaneous, etc) may impact dose amount and/or required dose amount may impact route of delivery. For example, where particularly high concentrations of an agent within a particular site or location are of interest, focused delivery may be desired and/or useful. Other factors to be considered when optimizing routes and/or dosing schedule for a given therapeutic regimen may include, for example, the particular cancer being treated (e.g., type, stage, location, etc.), the clinical condition of a subject (e.g., age, overall health, etc.), the presence or absence of combination therapy, and other factors known to medical practitioners. The pharmaceutical compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. Preferably, such a composition can further comprise a pharmaceutically acceptable carrier or excipient for use in the treatment of cancer that that is compatible with a given method and/or site of administration, for instance for parenteral (e.g. sub-cutaneous, intradermal, or intravenous injection), intratumoral, or peritumoral administration. As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

In some embodiments the anti-CD25 antibody may be part of a combination therapy with other therapautic agents. Therefore, a second aspect of the invention provides an anti-CD25 antibody for use in the treatment of AML or DLBCL, wherein the anti-CD25 antibody is administered in combination with one or more further therapeutic agents. A third aspect of the invention provides a combination of an anti-CD25 antibody and one or more further therapeutic agents for use in the treatment of AML or DLBCL. The anti-CD25 antibody and the further therapeutic agent are for separate, simultaneous or sequential administration.

The anti-CD25 antibody may be administered in combination with co-stimulatory antibodies, chemotherapy and/or radiotherapy (by applying irradiation externally to the body or by administering radio-conjugated compounds), cytokine-based therapy, targeted therapy, a vaccine, or an adjuvant, or any combination thereof. “In combination” may refer to administration of the additional therapy before, at the same time as or after administration of the anti-CD25 antibody. The anti-CD25 antibody and further therapeutic agent can be for separate, simultaneous or sequential administration.

The anti-CD25 antibody and other therapeutic agent may be administered via the same or different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

In some embodiments the other therapeutic agent may be selected from one or more FLT3 inhibitors (e.g. gilteritinib, midostaurin, sorafenib, quizartinib, crenolanib), BCL-2 inhibitors (e.g. venetoclax), IDH inhibitors (e.g. enasidenib, ivosidenib), hypomethylating agents (e.g. azacitidine, decitabine), further antibodies (e.g. a CD33 antibody such as gemtuzumab ozogamicin) and combinations thereof. In some embodiments the anti-CD25 antibody may be used in combination with anthracyclines-cytarabine regimens (such as a daunorubicin and cytarabine regime), optionally with further therapeutic agents. In some embodiment, the antibody is for use in combination with a BCL-2 inhibitor and a hypomethylating agent. In some embodiments the anti-CD25 antibody is used in combination with venetoclax, optionally with a further therapeutic agent such as azacitidine.

Other therapeutic agents include but is not limited to other chemotherapeutic agents such as a cytotoxic drug. A chemotherapeutic agent includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IFN-γ, IL-2, IL-12, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

In some embodiments the other therapeutic agent may be an immune checkpoint inhibitor. In some embodiments the invention also provides treating AML with the anti-CD25 antibody in combination with at least one immune checkpoint inhibitor. As used herein, “immune checkpoint” or “immune checkpoint protein” refer to proteins belonging to inhibitory pathways in the immune system, in particular for the modulation of T-cell responses. Under normal physiological conditions, immune checkpoints are crucial to preventing autoimmunity, especially during a response to a pathogen. Cancer cells can alter the regulation of the expression of immune checkpoint proteins in order to avoid immune surveillance.

Examples of immune checkpoint proteins include but are not limited to PD-1, CTLA-4, BTLA, KIR, LAG3, TIGIT, CD155, B7H3, B7H4, VISTA and TIM3, and also OX40, GITR, ICOS, 4-1BB and HVEM. Immune checkpoint proteins may also refer to proteins which bind to other immune checkpoint proteins. Such proteins include PD-L1, PD-L2, CD80, CD86, HVEM, LLT1, and GAL9. Immune checkpoint inhibitors may inhibit immune checkpoint proteins. For example, the immune checkpoint inhibitors may be antibodies that specifically bind to an immune checkpoint inhibitor, or they may be other antagonists of the immune checkpoint proteins.

In some embodiments of the invention the immune checkpoint protein is PD-1 or PD-L1 and the immune checkpoint inhibitor may be an inhibitor of PD-1 or PD-L1, i.e. a PD-1 or PD-L1 antagonist. In a further embodiment the immune checkpoint inhibitor interferes with PD-1/PD-L1 interactions via anti-PD-1 or anti PD-L1 antibodies. Anti-PD-1 antibodies known in the art include Nivolumab and Pembrolizumab. Anti-PD-L1 antibodies include antibodies, such as Atezolizumab (MPDL3280A).

In some embodiments the other therapeutic agent may be a cancer vaccine. A further embodiment of the invention provides treating AML with the anti-CD25 antibody in combination with a cancer vaccine. “Cancer vaccines” as used herein refer to therapeutic cancer vaccines administrated to cancer patients and designed to eradicate cancer cells through strengthening patient's own immune responses. Cancer vaccines include tumour cell vaccines (autologous and allogenic), dendritic cell vaccines (ex vivo generated and peptide-activated), protein/peptide-based cancer vaccines and genetic vaccines (DNA, RNA and viral based vaccines). Accordingly, therapeutic cancer vaccines, in principle, may be utilized to inhibit further growth of advanced cancers and/or relapsed tumours that are refractory to conventional therapies, such as surgery, radiation therapy and chemotherapy. Tumour cell based vaccines (autologous and allogeneic) include those genetically modified to secrete soluble immune stimulatory agents such as cytokines (IL-2, IFN-g, IL12, GMCSF, FLT3L), single chain Fv antibodies against immune modulatory receptors (PD-1, CTLA-4, GITR, ICOS, OX40, 4-1BB) and/or to express on their membrane the ligand for immune-stimulatory receptors such as ICOS-ligand, 4-1BB ligand, GITR-ligand, and/or OX40 ligand amongst others. In some embodiments the cancer vaccine may be a GVAX anti-tumour vaccine.

In some embodiments the combination therapy does not comprise the administration of a cancer vaccine in combination with the anti-CD25 antibody.

In some embodiments the anti-CD25 antibody is not conjugated to another therapeutic agent, for example the anti-CD25 antibody is not in the form of an antibody-drug conjugate. In some embodiments the anti-CD25 antibody is not camidanlumab tesirine (ADCT-301). Camidanlumab tesirine is the anti-CD25 antibody known as HuMax®-TAC conjugated through a dipeptide cleavable linker to pyrrolobenzodiazepine (PBD) dimer.

In some embodiments of the invention the anti-CD25 antibody is administered as a monotherapy. For example, when the anti-CD25 antibody is administered as a monotherapy, the anti-CD25 antibody is the only therapeutically active agent that is administered, for example the only therapeutically active agent that is administered to treat AML or DLBCL.

A fourth aspect of the invention provides a method of treating AML or DLBCL in a subject comprising administrating to the subject an effective amount of an anti-CD25 antibody.

A fifth aspect of the invention provides the use of anti-CD25 antibody in the manufacture of a medicament for the treatment of AML or DLBCL. The anti-CD25 antibody for these further aspects of the invention can be an anti-CD25 antibody as described for the first aspect.

The methods of treating AML can further comprise administration of one or more further therapeutic agents. In some embodiments the method further comprises administrating one or more immune check point inhibitors, cancer vaccines, FLT3 inhibitors, BCL-2 inhibitors, IDH inhibitors, hypomethylating agents, further antibodies and combinations thereof, or administrating in combination with an anthracycline-cytarabine regime, as described above. The further therapeutic agent may be administered separate, simultaneous or sequential.

A sixth aspect of the invention provides the use of an anti-CD25 antibody and a further therapeutic agent in the manufacture of a medicament for the treatment of AML or DLBCL, wherein the anti-CD25 antibody and further therapeutic agent are for separate, simultaneous or sequential administration.

The further therapeutic agent for the fourth, fifth and sixth aspects invention can be defined for the first, second and third aspect of the invention.

The invention also relates to selecting a patient having acute myeloid leukemia (AML) for treatment with an anti-CD25 antibody. A seventh aspect of the invention provides a method for selecting a patient having AML for treatment with an anti-CD25 antibody comprising determining the expression level of CD25 on target cells in a sample from the patient, wherein if the cells have an expression level above about 900 CD25 molecules per cell the patient is suitable for treatment with the antibody.

The method can be used to determine whether a patient will be suitable for treatment with an anti-CD25 antibody. Where the target cells from the patient have an expression level above about 900, preferably above 1000 CD25 molecules per cell, the patent will be suitable for treatment with an anti-CD25 antibody. The method can then further comprise the step of administering an anti-CD25 antibody to the patient.

The method can further comprise the step of determining whether functional FcR+ effector cells are present in the sample.

The method can further comprise taking a sample from the subject. The sample may be a biological tissue or fluid sample from the subject. In some embodiments the sample is a bone marrow sample from the patient.

The expression level of CD25 on target cells can be determined by methods known in the art, including for example flow cytometry as discussed in the Examples.

AML patients with the FLT3 internal tandem duplication (FLT3-ITD) mutation are associated with a poor prognosis, and an increased risk of relapse (Dohner et al, 2017, Blood 129(4) 424-447). The FLT-ITD mutation and involves tandem duplication of at least 3 to greater than 1000 nucleotides within the juxtamembrane domain of FLT3. The inventors have surprisingly further found that AML patients bearing a FLT3-ITD mutation as compared to FLT3 wildtype showed a strong prevalence of CD25 expressing AML cells. Therefore, patients with the FLT3-ITD mutation can be targeted for anti-CD25 antibody therapy. The presence of the FLT3-ITD mutation can also be used as a biomarker to identify, diagnose and/or predict whether an AML patient would benefit from anti-CD25 antibody therapy, alone or in combination with other therapeutic agents to treat AML.

Accordingly, an eighth aspect of the invention comprises a method for selecting a patient having acute myeloid leukemia for treatment with an anti-CD25 antibody, the method comprising determining the presence or absence of an FLT3-ITD mutation in a sample from the patient, wherein if the mutation is present in the sample the patient is suitable for treatment with the antibody. The method can be used to identifying whether the patient would be particularly suitable for anti-CD25 antibody therapy.

If the patient is determined to have an FLT3-IRD mutation the method may further comprise administering and anti-CD25 antibody to the patient. The anti-CD25 antibody can be as defined above for other aspects of the invention.

A ninth aspect of the invention provides a method for predicting the response of an AML patient to treatment with an anti-CD25 antibody, the method comprising determining the presence or absence of an FLT3-ITD mutation in a sample from the patient, wherein the presence of the mutation in the sample is indicative for a patient who will respond to treatment with the anti-CD25 antibody.

In a tenth aspect the invention provides a method of treating acute myeloid leukemia in a subject comprising administrating to the subject an effective amount of an anti-CD25 antibody, wherein the subject comprises the presence of an FLT3-ITD mutation. The method can also comprise determining the presence of an FLT3-ITD mutation in a sample from a patient with AML.

When the patient is diagnosed with AML, the patient may be diagnosed with AML characterised as having a FLT3-ITD mutation. Dohner et al, 2017, Blood 129 (4) 424-447. If the patient is diagnosed with AML characterised by the presence of a FLT3-ITD mutation, an anti-CD25 antibody as described herein may be particular useful in the treatment of AML.

The presence or absence of the FLT3-ITD mutation can be determined by methods known in the art. See for example Spencer DH et al, 2013 Journal Molecular Diagnostics, vol. 15 (1), 81-83, Engen C et al, 2021, Molecular Oncology, vol. 15, 2300-2317. For example, the presence or absence of the mutation can be determined by a method selected from the group of DNA sequencing and mutation screening technology.

The sample may be a blood or bone marrow sample from the patient. In some embodiments the method of predicting or selecting are in vitro methods.

An eleventh aspect of the invention provides a method for preventing or reducing the risk of relapse in an AML patient, the method comprising administering an anti-CD25 antibody to the patient. The anti-CD25 antibody can be as defined above for other aspects of the invention.

The anti-CD25 antibody may be used to target LSC (leukemic stem cells) and/or immature AML blasts or cells with a progenitor phenotype to help prevent or reduce the risk of relapse.

The method may further comprise administration of one of more further therapeutic agents. The one of more further therapautic agent can be as defined for the first, second and third aspect of the invention. In one embodiment the further therapeutic agent is a FLT3 inhibitor. In a further embodiment the one of more further therapeutic agents is the combination of a BCL-2 inhibitor and a hypomethylating agent, for example the combination of venetoclax and azacitidine. The anti-CD25 antibody and the further therapeutic agents can be for separate, simultaneous or sequential administration.

A twelfth aspect of the invention comprises a method for treating AML in a patient who has undergone, BCL-2 inhibitor-hypomethylating agent combination treatment, the method comprising administering an anti-CD25 antibody to the patient. The anti-CD25 antibody can be as described above. In one embodiment the BCL-2 inhibitor-hypomethylating agent combination treatment comprises venetoclax-azacitidine combination.

The inventors have found that AML patients treated with a BCL-2 inhibitor and a hypomethylating agent, such as the combination of venetoclax and azacitidine still displayed detectable levels of CD25+ AML cells. Therefore, further treating the AML patients with an anti-CD25 antibody, separate, simultaneous or by sequential administration, may help prevent relapse and disease progression.

The invention also provides for an anti-CD25 antibody for use in the methods of the above further aspects of the invention.

Aspects and embodiments described herein with the term “comprising” may include other features or steps within the scope. It is also understood that aspects and embodiments described as “comprising” also describes aspect and embodiments wherein the term “comprising” is replaced by the term “consisting essentially of” or “consisting of”.

The phrase “selected from the group comprising” may be substituted with the phrase “selected from the group consisting of” and vice versa, wherever they occur herein.

It is also understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

The invention will now be further described by way of the following Examples, which are meant to serve to assist one of ordinary skill in the art in carrying out the invention and are not intended in any way to limit the scope of the invention, with reference to the Figures.

### EXAMPLES

### Example 1

### Cells and Antibodies

Pfeiffer tumor cells (ATCC), an established large B-cell lymphoma suspension cell line, were cultured in RPMI 1640 medium (Gibco) containing 20% FBS (Gibco), 1×Glutamax (Gibco), 1×Non Essential Amino Acids (Gibco) and 1% Sodium Pyruvate (Gibco). EOL-1 tumor cells (DSMZ), an established acute myeloid leukemia suspension cell lines, were cultured in RPMI 1640 medium (Gibco) containing 10% FBS (Gibco), 1×Glutamax (Gibco), 1×Non Essential Amino Acids (Gibco) and 1% Sodium Pyruvate (Gibco).

AML22 cells originated from an AML patient and were expanded by implantation in NSG mice. Since these patient-derived cells cannot be cultured in vitro, they were thawed and used directly on the day of the assay.

In vitro induced Treg (iTreg) cells were differentiated from naive CD4+ T cells by activation with Dynabeads Human T-Activator CD3/CD28 (1×10⁶ beads/mL, bead-to-cell ratio 1:1, Gibco) for 10 days in iTreg medium consisting of X-Vivo 15 (Lonza) supplemented with 10% heat-inactivated human AB serum (Sigma), 1×Glutamax (Gibco), N-acetylcysteine (2 mg/mL, Sigma), 1% sodium pyruvate (Gibco), 1×HEPES (Gibco), 1×Non Essential Amino Acids (Gibco), 50 μM 2-mercaptoethanol (Thermo Fisher Scientific), Proleukin/Aldesleukin (300 U/mL, Novartis), 10 ng/ml recombinant human transforming growth factor-β1 (R&D Systems) and 100 ng/ml rapamycin (Sigma). Naive CD4+ T cells were isolated from human PBMCs using the human naive CD4+ T-cell isolation kit II (Miltenyi Biotec). Purity of iTreg was confirmed by flow cytometry analysis of human CD3, CD4, CD25 and FoxP3 co-expression (>90%). Cryopreserved iTregs were thawed and used directly on the day of the assay.

CD25 Mab, also referred to as RG6292 is an afucosylated human IgG1 mAb produced using the GlymaxX technology that confers an enhanced ADCC capacity of CD25 expressing target cells. Human IgG1 isotype control antibody was purchased from Biolegend.

### Human PBMC and NK Cell Isolation

Human PBMCs from healthy donors were isolated from buffy coats (Zurich Blood Donation Center in accordance with the Declaration of Helsinki) using standard density-gradien isolation over Histopaque-1077 (Sigma-Aldrich). NK cells were isolated using the human NK cell isolation kit (Miltenyi Biotec) and activated overnight in RPMI 1640 medium (Gibco) containing 10% FCS (Gibco), 1×Glutamax (Gibco) and Proleukin/Aldesleukin (100 U/mL, Novartis).

ADCC assay with Flow Cytometry Readout

Target cells (Pfeiffer, EOL-1, AML22, iTregs) were mixed with activated primary NK cells at a 2:1 effector to target cell ratio (80,000 NK cells and 40,000 target cells per well) in assay medium (RPMI 1640 (Gibco) containing 2% FBS (Gibco) and 1×Glutamax (Gibco)). Compounds (CD25 Mab or isotype control) were added to the U bottom 96-well plate (TPP) at a 7-fold dilution series, with a starting final concentration of 21 μg/mL. Assay plates were placed on an orbital shaker for 5 minutes at 300 rpm to mix cells and antibodies. After 15 minutes of pre-incubation at room temperature, the plates were incubated at 37° C./5% CO2 for 17 hours.

Cells were stained with FSV440UV viability dye diluted in PBS (BD) for 10 minutes at room temperature. Cells were washed with FACS buffer (PBS containing 0.1% BSA) and stained with fluorescent dye-conjugated antibodies against human CD16 (3G8), CD45 (HI3O), CD34 (8G12), CD56 (5.1H11), CD3 (UCHT1), CD20 (2H7), CD117 (YB5.B8), CD25 (24212), CD123 (9F5), CD69 (FN50), CD4 (A161A1), CD127 (A019D5) in FACS buffer for 30 minutes at 4° C. After two washing steps with FACS buffer, cells were fixed and permeabilized using FoxP3 Transcription Factor Staining Set (eBioscience) for 60 minutes at room temperature.

For intra-nuclear staining, cells were subsequently stained against human FoxP3 (259D), Bcl-2 (Bcl-2/100) and Ki-67 (Ki67) in 1×PERM buffer for 45 minutes at room temperature. Samples were washed twice with 1×PERM buffer and resuspended in FACS buffer for acquisition. All antibodies used for flow cytometry were from Biolegend, BD or R&D Systems.

Samples were acquired with a 5-laser A5 Symphony instrument (BD). Data analysis was performed with FlowJo v10.6.2 and Prism 8 (GraphPad software).

ADCC Assay with Luminescence Readout

Target cells (Pfeiffer, EOL-1, AML22) were mixed with activated primary NK cells at a 2:1 effector to target cell ratio (20,000 NK cells and 10,000 target cells per well) in assay medium (RPMI 1640 (Gibco) containing 2% FBS (Gibco) and 1×Glutamax (Gibco)). Compounds (CD25 Mab or isotype control) were added to a white 384-well flat bottom tissue culture plate (Falcon) at a 7-fold dilution series, with a starting final concentration of 21 μg/mL. Assay plates were placed on an orbital shaker for 5 minutes at 300 rpm to mix cells and antibodies. After 15 minutes of pre-incubation at room temperature, the plates were incubated at 37° C./5% CO₂ for 16-20 hours.

Assay plates were equilibrated at room temperature for approximately 15 minutes without lid. Cytotoxicity was measured using the CytoTox-Glo™ Cytotoxicity Assay (Promega) according to the manufacturers' instructions. Briefly, reconstituted AAF-Glo™ reagent wasυ added to each well at a 1 to 4 dilution (final volume 40 μl/well). Assay plates were incubated for 15-60 minutes at room temperature, followed by luminescence measurement using Tecan Spark 10M luminescence plate reader (500 ms integration time). Data analysis was performed with Prism 8 (GraphPad software).

### Quantification of CD25 Density by Flow Cytometry

BD quantibrite™ Beads were used to estimate antibodies bound per cell (ABC) which is equivalent to the number of PE molecules per cell if PE:mAb ratio is 1:1. If monovalent binding of the PE-conjugated anti-human CD25 antibody is assumed, the number of CD25molecules is equivalent to the number of PE molecules on the cell surface.

Cellular assay samples and beads were acquired with the same instrument settings on a 5-later Symphony instrument (BD). Analysis of flow cytometry data was performed in FlowJo v10.6 (Tree Star) and Prism 8 (GraphPad software). Linear regression of Log10 PE molecules per bead against Log10 fluoresence as well as interpolation of number of PE molecules per cell were determined according to the manufacturer's instructions.

CD25 density was quantified on the four CD25 expressing target cells.

### Results

To assess the capacity of CD25 Mab to kill AML cells with a lower level of CD25 expression, an Antibody Dependent Cellular Cytotoxicity (ADCC) assay was performed comparing target cells with a range of CD25 expression levels. The CD25 density (number of molecules per cell) was quantified using BD quantibrite™ beads. If monovalent binding of the antibody is assumed, the number of CD25 molecules is equivalent to the number of PE molecules on the cell surface.

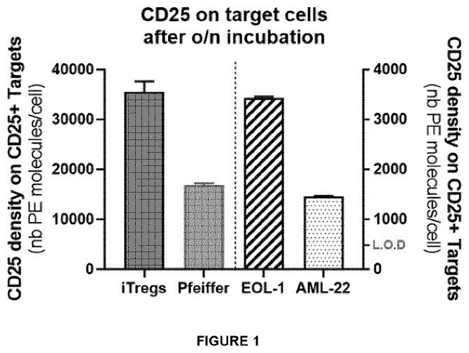

As shown in FIG. 1, iTregs and the DLBCL cell line Pfeiffer express relatively high levels of CD25, whereas AML cell line EOL-1 and patient derived AML22 cells harbor a lower number of CD25 receptors on their surface. These results were obtained 17 hours after incubation of target cells alone in ADCC assay medium.

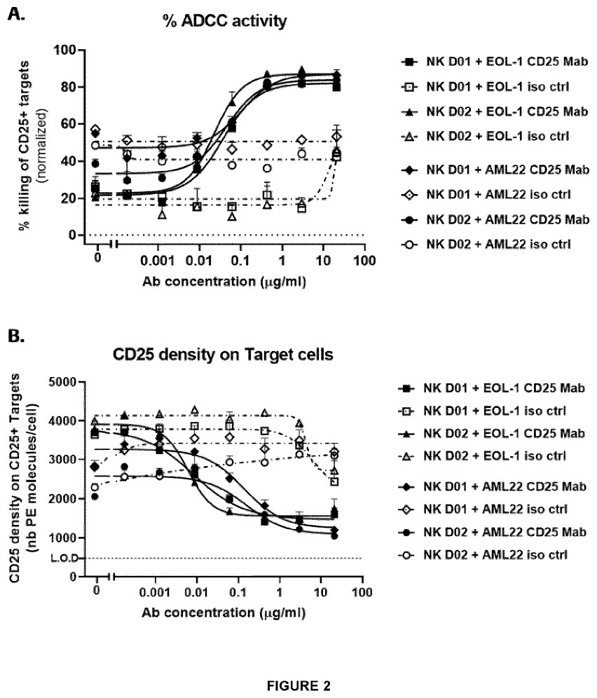

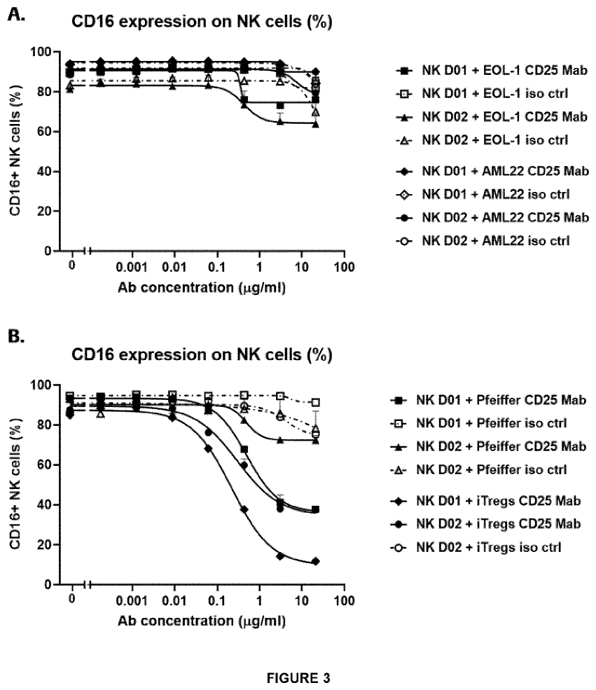

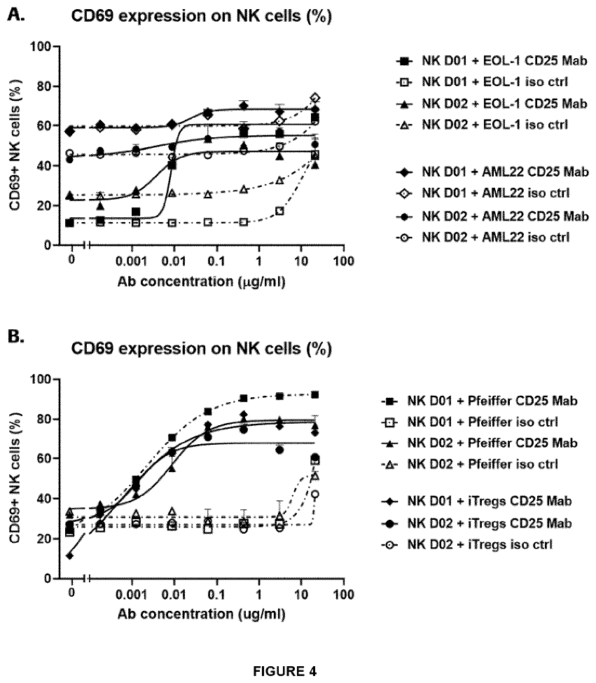

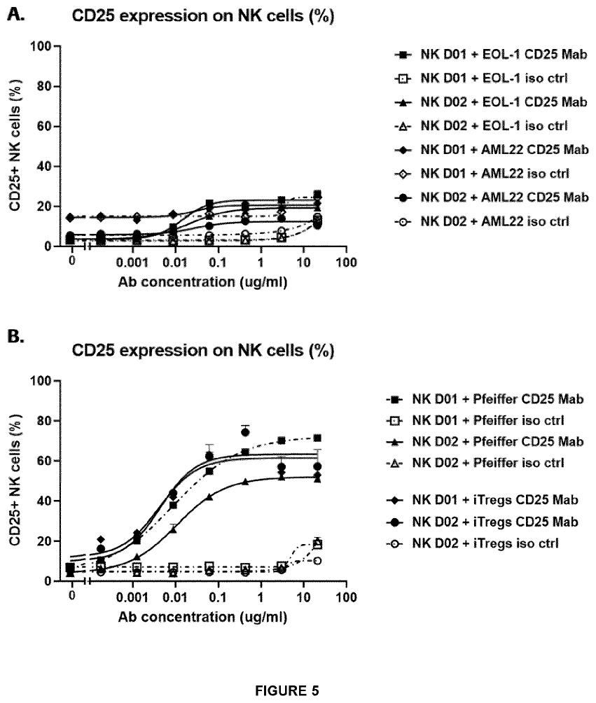

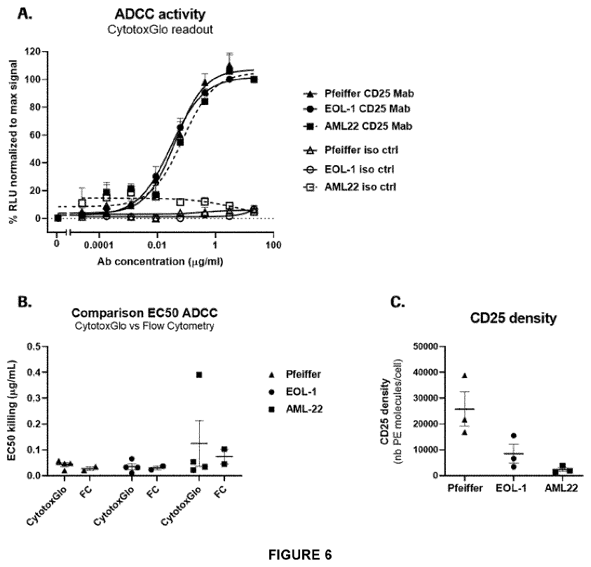

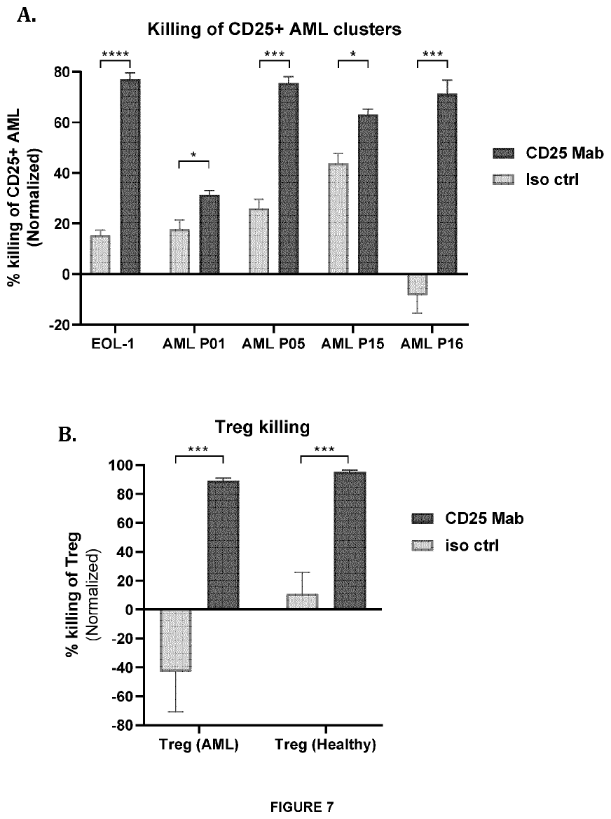

Despite the fact that CD25 Mab features an avidity driven binding to CD25 and as a consequence preferentially triggers the killing of cells with high levels of CD25 expression (Solomon et al, Nat Cancer (2020) vol 1(12) p1153-1166), the ADCC capacity of CD25 Mab with EOL-1 and AML-22 as target cells were comparable to that of CD25 high target cells. More than 80% of the target cells were killed by NK cells after 17 hours of co-incubation with CD25 Mab (FIG. 2A) whereas no specific killing was observed with the isotype control antibody. The increase in EOL-1 cell line killing observed at the highest isotype control concentration (21 μg/mL) can likely be attributed to unspecific activity. While the EC50 values were similar (˜0.03 μg/mL) for the two cell lines tested, baseline killing in the absence of compounds differed. Indeed, 40-50% of AML-22 were killed by activated NK cells as compared to 20% of EOL-1 cells. AML-22 cells are primary cells that rapidly drop in viability when cultured in vitro and we hypothesize that they may express molecules that render them more susceptible to NK cell mediated killing.

The CD25 density on the target cells that remained viable at the end of the 17 hours ADCC assay (FIG. 2B) was also assessed. It was found that at CD25 Mab concentrations above 0.4 μg/mL, target cells expressed 1,000-1,500 CD25 molecules per cell. Moreover, peripheral and intratumoral human conventional CD4 and CD8 T cells with CD25 expression levels of ˜800 and 400 receptors per cell, respectively, do not seem to be targeted for depletion by CD25 Mab (data not shown). Therefore, this indicates that the threshold of CD25 expression on target cells needed to trigger killing by CD25 Mab is likely around 1,000 receptors per cell. As CD25 density in primary human AML bone marrow samples (CD25+ AML blasts and Tregs) ranged from 1,000 to 4,000 (data not shown), it is anticipated to see depletion of those cells.

The expression of functional markers on NK cells after target engagement through the binding of the antibody Fc portion to the FcγRIIIa (CD16) receptor expressed on CD56dim NK cells was investigated. This subset of NK cells represents the most prevalent population (90% of NK cells) in human peripheral blood and is, as opposed to the CD56brightCD16neg NK cell subset, a potent ADCC mediator.

A modest down-regulation of CD16 was observed on NK cells after engagement with low CD25 density EOL-1 and AML-22 target cells (FIG. 3A). In contrast, CD16 expression was strongly decreased in a CD25 Mab dose-dependent manner after effector cell co-incubation with high CD25 density Pfeiffer and iTregs target cells (FIG. 3B). Therefore, the results indicate that the strength of CD16 down-regulation correlates with the CD25 density on the target cells.

In addition, the functionality of NK cells can be measured by the upregulation of the activation markers CD69 and CD25. The expression of CD69 was increased in a dose dependent fashion after treatment with CD25 Mab in all tested target cells. Similar to the observation of a high baseline killing of AML22 target cells, CD69 baseline expression was also increased. Indeed, 45-60% of NK cells expressed CD69 when co-incubated with AML22 and in the absence of drug compound (FIG. 4A). Taken together, these results suggest that AML22 target cells express receptors or secrete factors that induce a strong NK cell baseline activation level. On the other hand, only 20% of NK cells expressed CD69 at baseline when in contact with the other three target cells, and the expression increased up to 80% after CD25 Mab treatment (FIG. 4B).

An upregulation of CD25 on NK cells was also observed, and the strength of the upregulation was proportional to the CD25 density on the target cells. As shown in FIG. 5A, NK cells co-incubated with low density CD25+ target cells (AML22 and EOL-1) displayed a modest 20% increase in CD25 expression. In contrast, up to 60% of NK cells treated with CD25 Mab expressed CD25 when exposed to target cells with a high CD25 density (FIG. 5B). Of note, despite the increase in CD25 expression, NK cell counts remained unaffected, indicating that there is no sign of fratricide (data not shown).

In order to increase the confidence in the results obtained by flow cytometry, an ADCC assay was performed using four NK cell donors and a luminescence readout (CytoTox-Glo™ Cytotoxicity Assay). Consistent and comparable killing activity was observed with the three target cells (AML22, Pfeiffer, EOL-1) as shown in FIG. 6A. The EC50 values calculated from dose-response curves demonstrated that similar results were obtained between flow cytometry-based and luminescence-based ADCC readouts. For instance, EC50 value with EOL-1 target cells was 0.035 μg/mL (±0.011 SEM) using CytoTox-Glo™ and 0.030 (±0.007 SEM) μg/mL by flow cytometry (FIG. 6B). CD25 Mab demonstrated potent killing activity across target cells with a CD25 density that ranges from 1,500 to 38,000 receptors per well (FIG. 6C). Since CD25 density in human samples lies within this range of expression (data not shown), we expect efficient killing of target cells in human PBMC and tumors of AML and DLBCL patients, providing that functional FcR+ effectors cells are present. These experiments show the direct killing of CD25+ AML and DLBCL cells. The antibody was able to deplete CD25+ malignant cells with a wide range of CD25 expression level. Since the expression level in human samples is within this range and the indirect effect of CD25 Mab through Treg depletion has already been demonstrated, we anticipate a dual mode of action of CD25 Mab to be involved in the treatment of AML and DLBCL. CD25 Mab can deplete suppressive Tregs and have a direct cytotoxicity effect on the CD25+ malignant cells of AML and DLBCL.

### Example 2

### Primary Human Samples

Human peripheral blood mononuclear cells (PBMC) and bone marrow mononuclear cells (BMMC) of AML patients were purchased from Discovery Life Sciences. Samples were collected under the approval of the relevant Institutional Review Board (IRB) or Ethics Committee. Healthy donor (HD) PBMC were isolated from buffy coats (Zurich Blood Donation Center) using standard density-gradient centrifugation. All human samples were collected in accordance with the Declaration of Helsinki from HD or patients who had provided written informed consent.

### NK Cell Isolation

NK cells were isolated using the human NK cell isolation kit (Miltenyi Biotec) activated overnight in RPMI 1640 medium (Gibco) containing 10% FCS (Gibco), 1×Glutamax (Gibco) and Proleukin/Aldesleukin (100 U/mL, Novartis).

### ADCC Assay

EOL-1 positive control cell line or AML patient samples containing CD25 expressing target cells were mixed with activated primary NK cells at a 2 to 1 effector to target cell ratio (80,000 NK cells and 40,000 target cells per well) in assay medium (RPMI 1640 (Gibco) containing 2% FBS (Gibco) and 1×Glutamax (Gibco)). Compounds (CD25 Mab (RG6292) or isotype control (Human IgG1 isotype control antibody, Biolegend, QA16A12)) were added to U bottom 96-well plate (TPP) at a concentration of 10 μg/mL. Assay plates were placed on an orbital shaker for 5 minutes at 300 rpm to mix cells and antibodies. After 15 minutes of pre-incubation at room temperature, samples were incubated at 37° C./5% CO₂ for 20 hours. Flow cytometric readout was performed as described in the section below. Precision Count Beads™ (Biolegend) were added before sample acquisition and killing activity calculated based on absolute counts (number of cells/μl) normalized to the number of target cells in the absence of allogeneic effector NK cells and compounds.

### Results

Having demonstrated cytotoxicity of CD25 Mab on EOL-1 cell line and AML22 cells (Example 1), we sought to assess its functional activity with AML patient material. For this purpose, we selected four patients with the highest frequency of CD25+ AML cells and performed an ex vivo ADCC assay. We showed specific killing of CD25+ AML cells in all samples at saturating antibody concentration (FIG. 7(A)). Moreover, we assessed Treg killing in the same experimental setup and found that both AML and HD Tregs were efficiently depleted by CD25 Mab treatment (FIG. 7(B)). Taken together, these results provide a proof of concept of CD25 Mab's dual mode of action in AML patient samples using HD NK cells.

### Example 3

### Flow Cytometry Panel Design, Staining and Acquisition

We followed recent guidelines (Liechti et al., 2021, Nat Immunol 22, 1190-1197) for panel design and optimal validation. Of note, all antibodies were titrated and a combination of Fluorescence-minus-one (FMO) controls as well as biological controls (cell populations lacking one or a set of markers) were utilized to validate the panels.

Cryopreserved AML patient and HD samples were thawed in DMEM/F-12 medium (Gibco) containing 10% FBS (Gibco). Cells were incubated with Human TruStain FcX (Biolegend) and stained with Zombie NIR (Biolegend) viability dye diluted in PBS. Then, cells were stained with fluorochrome-conjugated antibodies against surface antigens (HLA-DR (G46-6), CD16 (3G8), CD45 (HI30), CD33 (P67.6), CD45RA (HI100), CD34 (8G12), CD56 (5.1H11), CD3 (UCHT1), CD19 (HIB19), CD117 (YB5.B8), CD25 (24212), CD123 (9F5), CD69 (FN50), CD4 (A161A1), CD8a (RPA-T8), CD71 (M-A712), CD127 (A019D5), CD14, CD38 (HIT2), CD235A (HIR2), CLEC12A (50C1), PD-1 (EH12.1), TIM3 (7D3)) in staining buffer containing FACS buffer (PBS containing 0.1% BSA) and Brilliant Stain buffer (BD). Cells were fixed and permeabilized using FoxP3 Transcription Factor Staining Set (eBioscience) and subsequently stained against intra-cellular antigens (FoxP3 (259D), Bcl-2 (Bcl-2/100) and Ki-67 (Ki67)) in 1×PERM buffer. Samples were acquired with a 5-laser Aurora spectral cytometer (Cytek).

Pre-processing steps (applying of compensation matrix, gating on live single cells) were performed with SpectroFlo® software. Unmixed files were checked individually and manual spillover compensation adjusted when necessary using the integrated software. Of note, only minor modifications were needed. Events of FCS files that passed all quality control steps were exported for further computational analysis with R.

### Computational Flow Cytometry Data Analysis

We used a patient-centric approach to identify CD25+ AML clusters and Tregs and applied the computational analysis workflow described below for each patient separately. FCS files were loaded into R and processed as described in the vignette of the flowCore R package. Logicle transformation was applied onto the expression matrix. Density plots for each marker were visually inspected and thresholds defined to allow for the quantification of positive and negative expression, similarly to positivity cutoffs assigned by traditional manual gating.

Dimensionality reduction was performed using the uniform manifold approximation and projection (UMAP) algorithm as part of the uwot R package. Unsupervised clustering was performed in the high-dimensional space with PhenoGraph (Levine et al., 2015, Cell 162, 184-197) and results thereof visualized on the UMAP plot as a color overlay. Algorithm-generated clusters were merged and annotated manually based on marker expression in order to arrive at biologically meaningful cell populations. Downstream analysis reported cell abundance, marker expression as well as fractions of positive cells where appropriate. Cells negative for CD45 expression and all other markers present in the panel (non-immune cells) as well as very small clusters (<0.05%) were filtered out.

CD25+ cell populations identified by computational analysis were validated by manual gating. Figures were generated with Prism v8.4.2 (GraphPad Software).

### Results

Internal tandem duplications (ITD) in the FLT3 gene are present in about 25% of AML patients, are associated with a poor prognosis and an increased risk of relapse (Dohner et al., 2010, Blood 115, 453-474).

Using high dimensional flow cytometric analysis of AML patient samples, we demonstrated that the presence of FLT3-ITD mutation led to a strong enrichment in the prevalence of CD25+ AML cells (data not shown), as previously reported by others (Angelini et al., 2015, Clin Cancer Res 21, 3977-3985; Aref et al., 2020, Leuk Res Rep 13, 100203).

Moreover, we observed that BCL-2 was highly expressed on CD25+ AML clusters with an immature phenotype (data not shown). This finding is in line with prior reports that showed high BCL-2 expression in the leukemic stem cell compartment (Lagadinou et al., 2013; Cell Stem Cell 12, 329-341, Renders et al., 2021, Blood 138, 3469-3469). Interestingly, CD25+ AML clusters were detected in all four patients treated with a hypomethylating agent in combination with venetoclax (HMA-VEN).

Therefore, these results support the treatment of AML patients having FLT3-ITD mutation with an anti-CD25 antibody. These results also support the use an anti-CD25 antibody as combinatorial treatment with, for instance, FLT3 inhibitors or with BCL-2 inhibitors, e.g venetoclax, in the treatment of AML, in particular to reduce the risk of relapse.

All publications mentioned in the above specification are herein incorporated by reference. Various modifications and variations of the described methods and system of the invention will be apparent to those skilled in the art without departing from the scope and spirit of the invention. Although the invention has been described in connection to specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

A summary of sequences referred to in the application is provided in the table below:

TABLE 2

| SEQ | | |
|---|---|---|
| ID | | |
| NO | Description of Antibody Sequences | Also referred to as: |
| 1 | MDSYLLMWGL LTFIMVPGCQ AELCDDDPPE IPHATFKAMA | Human CD25 sequence |
| | YKEGTMLNCE CKRGFRRIKS GSLYMLCTGN SSHSSWDNQC | |
| | QCTSSATRNT TKQVTPQPEE QKERKTTEMQ SPMQPVDQAS | |
| | LPGHCREPPP WENEATERIY HFVVGQMVYY QCVQGYRALH | |
| | RGPAESVCKM THGKTRWTQP QLICTGEMET SQFPGEEKPQ | |
| | ASPEGRPESE TSCLVTTTDF QIQTEMAATM ETSIFTTEYQ | |
| | VAVAGCVFLL ISVLLLSGLT WQRRQRKSRR TI | |
| 2 | GTFSSLAIS | CDR-H1 aCD25-a-686 |
| | | CDR-H1 aCD25-a-686-m1 |
| | | CDR-H1 aCD25-a-686-m3 |
| 3 | GTFSSLAIT | CDR-H1 aCD25-a-686-m2 |
| 4 | GTFSALAIS | CDR-H1 aCD25-a-686-m4 |
| 5 | GTFSSLAIF | CDR-H1 aCD25-a-686-m5 |
| 6 | GIIPIFGTANYAQKFQG | CDR-H2 aCD25-a-686 |
| 7 | AIIPVFGTASYAQKFQG | CDR-H2 aCD25-a-686-m1 |
| 8 | GIIPIFGDASYAQKFQG | CDR-H2 aCD25-a-686-m2 |
| 9 | GIIPIFGDANYAQKLQG | CDR-H2 aCD25-a-686-m3 |
| 10 | GIIPLFGRANYAQKFQG | CDR-H2 aCD25-a-686-m4 |
| 11 | GIIPVFGQANYAQKFQG | CDR-H2 aCD25-a-686-m5 |
| 12 | ARGGSVSGTLVDFDI | CDR-H3 aCD25-a-686 |
| | | CDR-H3 aCD25-a-686-m1 |
| | | CDR-H3 aCD25-a-686-m2 |
| | | CDR-H3 aCD25-a-686-m3 |
| | | CDR-H3 aCD25-a-686-m4 |
| | | CDR-H3 aCD25-a-686-m5 |
| 13 | RASQSISSWLA | CDR-L1 aCD25-a-686 |
| | | CDR-L1 aCD25-a-686-m1 |
| | | CDR-L1 aCD25-a-686-m2 |
| | | CDR-L1 aCD25-a-686-m3 |
| | | CDR-L1 aCD25-a-686-m4 |
| | | CDR-L1 aCD25-a-686-m5 |
| 14 | KASSLES | CDR-L2 aCD25-a-686 |
| | | CDR-L2 aCD25-a-686-m1 |
| | | CDR-L2 aCD25-a-686-m2 |
| | | CDR-L2 aCD25-a-686-m3 |
| | | CDR-L2 aCD25-a-686-m4 |
| | | CDR-L2 aCD25-a-686-m5 |
| 15 | QQYNIYPIT | CDR-L3 aCD25-a-686 |
| | | CDR-L3 aCD25-a-686-m1 |
| | | CDR-L3 aCD25-a-686-m2 |
| | | CDR-L3 aCD25-a-686-m3 |
| | | CDR-L3 aCD25-a-686-m4 |
| | | CDR-L3 aCD25-a-686-m5 |
| 16 | QVQLVQSGAEVKKPGSSVKVSCKASGGTESSLAISWVRQAPGQGL | VH aCD25-a-686 |
| | EWMGGIIPIFGTANYAQKFQGRVTITADESTSTAYMELSSLRSED | |
| | TAVYYCARGGSVSGTLVDFDIWGQGTMVTVSS | |
| 17 | QVQLVQSGAEVKKPGSSVKVSCKASGGTESSLAISWVRQAPGQGL | VH aCD25-a-686-m1 |
| | EWMGAIIPVFGTASYAQKFQGRVTITADESTSTAYMELSSLRSED | |
| | TAVYYCARGGSVSGTLVDFDIWGQGTMVTVSS | |
| 18 | QVQLVQSGAEVKKPGSSVKVSCKASGGTFSSLAITWVRQAPGQGL | VH aCD25-a-686-m2 |
| | EWMGGIIPIFGDASYAQKFQGRVTITADESTSTAYMELSSLRSED | |
| | TAVYYCARGGSVSGTLVDFDIWGQGTMVTVSS | |
| 19 | QVQLVQSGAEVKKPGSSVKVSCKASGGTFSSLAISWVRQAPGQGL | VH aCD25-a-686-m3 |
| | EWMGGIIPIFGDANYAQKLQGRVTMTTDTSTSTAYMELRSLRSDD | |
| | TAVYYCARGGSVSGTLVDEDIWGQGTMVTVSS | |
| 20 | QVQLVQSGAEVKKPGSSVKVSCKASGGTFSALAISWVRQAPGQGL | VH aCD25-a-686-m4 |
| | EWMGGIIPLFGRANYAQKFQGRVTITADESTSTAYMELSSLRSED | |
| | TAVYYCARGGSVSGTLVDFDIWGQGTMVTVSS | |
| 21 | QVQLVQSGAEVKKPGSSVKVSCKASGGTFSSLAIFWVRQAPGQGL | VH aCD25-a-686-m5 |
| | EWMGGIIPVFGQANYAQKFQGRVTITVDESTSTAYMELSSLRSED | |
| | TAVYYCARGGSVSGTLVDFDIWGQGTMVTVSS | |
| 22 | DIQMTQSPSTLSASVGDRVTITCRASQSISSWLAWYQQKPGKAPK | VL aCD25-a-686 |
| | LLIYKASSLESGVPSRFSGSGSGTEFTLTISSLQPDDFATYYCQQ | VL aCD25-a-686-m1 |
| | YNIYPITFGGGTKVEIK | VL aCD25-a-686-m2 |
| | | VL aCD25-a-686-m3 |
| | | VL aCD25-a-686-m4 |
| | | LC aCD25-a-686-m5 |
| 23 | YTFTSYYMH | CDR-H1 aCD25-a-674 |
| 24 | IINPSGGSTSYAQKFQG | CDR-H2 aCD25-a-674 |
| 25 | ARGGAEYIPAEYFQH | CDR-H3 aCD25-a-674 |
| 26 | RASQSVSSYLA | CDR-L1 aCD25-a-674 |
| 27 | DASNRAT | CDR-L2 aCD25-a-674 |
| 28 | QQRPFLPT | CDR-L3 aCD25-a-674 |
| 29 | QVQLVQSGAEVKKPGASVKVSCKASGYTFTSYYMHWVRQAPGQGL | VH aCD25-a-674 |
| | EWMGIINPSGGSTSYAQKFQGRVTMTRDTSTSTVYMELSSLRSED | |
| | TAVYYCARGGAEYIPAEYFQHWGQGTLVTVSS | |
| 30 | EIVMTQSPATLSLSPGERATLSCRASQSVSSYLAWYQQKPGQAPR | VL aCD25-a-674 |
| | LLIYDASNRATGIPARFSGSGSGTDFTLTISSLEPEDFAVYYCQQ | |
| | RPFLPTFGGGTKVEIK | |
| 31 | FTFSSYGMH | CDR-H1 aCD25-a-646 |
| 32 | FTFASYGMH | CDR-H1 aCD25-a-646-m1 |
| 33 | FTFPSYGMH | CDR-H1 aCD25-a-646-m2 |
| | | CDR-H1 aCD25-a-646-m3 |
| | | CDR-H1 aCD25-a-646-m4 |
| | | CDR-H1 aCD25-a-646-m5 |
| 34 | VIWYDGSNKGYADSVKG | CDR-H2 aCD25-a-646 |
| 35 | VIWYDASTKYYADSVKG | CDR-H2 aCD25-a-646-m1 |
| | | CDR-H2 aCD25-a-646-m4 |
| 36 | VIWYDAINKYYADSVKG | CDR-H2 aCD25-a-646-m2 |
| 37 | VIWYDAVNKYYADSVKG | CDR-H2 aCD25-a-646-m3 |
| 38 | VIWYDALNKYYADSVKG | CDR-H2 aCD25-a-646-m5 |
| 39 | ARDLGYGDYAAHDY | CDR-H3 aCD25-a-646 |
| | | CDR-H3 aCD25-a-646-m1 |
| | | CDR-H3 aCD25-a-646-m2 |
| | | CDR-H3 aCD25-a-646-m3 |
| | | CDR-H3 aCD25-a-646-m4 |
| | | CDR-H3 aCD25-a-646-m5 |
| 40 | RASQSISSWLA | CDR-L1 aCD25-a-646 |
| | | CDR-L1 aCD25-a-646-m1 |
| | | CDR-L1 aCD25-a-646-m2 |
| | | CDR-L1 aCD25-a-646-m3 |
| | | CDR-L1 aCD25-a-646-m4 |
| | | CDR-L1 aCD25-a-646-m5 |
| 41 | KASSLES | CDR-L2 aCD25-a-646 |
| | | CDR-L2 aCD25-a-646-m1 |
| | | CDR-L2 aCD25-a-646-m2 |
| | | CDR-L2 aCD25-a-646-m3 |
| | | CDR-L2 aCD25-a-646-m4 |
| | | CDR-L2 aCD25-a-646-m5 |
| 42 | QQHNTHPYT | CDR-L3 aCD25-a-646 |
| | | CDR-L3 aCD25-a-646-m1 |
| | | CDR-L3 aCD25-a-646-m2 |
| | | CDR-L3 aCD25-a-646-m3 |
| | | CDR-L3 aCD25-a-646-m4 |
| | | CDR-L3 aCD25-a-646-m5 |
| 43 | QVQLVESGGGVVQPGRSLRLSCAASGFTFSSYGMHWVRQAPGKGL | VH aCD25-a-646 |
| | EWVAVIWYDGSNKGYADSVKGRFTISRDNSKNTLYLQMNSLRAED | |
| | TAVYYCARDLGYGDYAAHDYWGQGTLVTVSS | |
| 44 | EVQLVESGGGLVKPGGSLRLSCAASGFTFASYGMHWVRQAPGKGL | VH aCD25-a-646-m1 |
| | EWVAVIWYDASTKYYADSVKGRFTISRDNSKNTLYLQMNSLRAED | |
| | TAVYYCARDLGYGDYAAHDYWGQGTLVTVSS | |
| 45 | QVQLVESGGGLVQPGGSLRLSCAASGFTFPSYGMHWVRQAPGKGL | VH aCD25-a-646-m2 |
| | EWVAVIWYDAINKYYADSVKGRFTISRDNSKNTLYLQMNSLRAED | |
| | TAVYYCARDLGYGDYAAHDYWGQGTLVTVSS | |
| 46 | QVQLVESGGGVVQPGRSLRLSCAASGFTFPSYGMHWVRQAPGKGL | VH aCD25-a-646-m3 |
| | EWVAVIWYDAVNKYYADSVKGRFTISRDNSKNTLYLQMNSLRAED | |
| | TAVYYCARDLGYGDYAAHDYWGQGTLVTVSS | |
| 47 | QVQLVESGGGVVQPGRSLRLSCAASGFTFPSYGMHWVRQAPGKGL | VH aCD25-a-646-m4 |
| | EWVAVIWYDASTKYYADSVKGRFTISRDNSKNTLYLQMNSLRAED | |
| | TAVYYCARDLGYGDYAAHDYWGQGTLVTVSS | |
| 48 | EVQLLESGGGLVQPGGSLRLSCAASGFTFPSYGMHWVRQAPGKGL | VH aCD25-a-646-m5 |
| | EWVAVIWYDALNKYYADSVKGRFTISRDNSKNTLYLQMNSLRAED | |
| | TAVYYCARDLGYGDYAAHDYWGQGTLVTVSS | |
| 49 | DIQMTQSPSTLSASVGDRVTITCRASQSISSWLAWYQQKPGKAPK | VL aCD25-a-646-m1 |
| | LLIYKASSLESGVPSRFSGSGSGTEFTLTISSLQPDDFATYYCQQ | VL aCD25-a-646-m2 |
| | HNTHPYTFGGGTKVEIK | VL aCD25-a-646-m3 |
| | | VL aCD25-a-646-m4 |
| | | VL aCD25-a-646-m5 |
| 50 | QVQLVQSGAE VKKPGSSVKV SCKASGGTFS SLAISWVRQA | HC sequence of RG6292 |
| | PGQGLEWMGG IIPIFGTANY AQKFQGRVTI TADESTSTAY | |
| | MELSSLRSED TAVYYCARGG SVSGTLVDFD IWGQGTMVTV | |
| | SSASTKGPSV FPLAPSSKST SGGTAALGCL VKDYFPEPVT | |
| | VSWNSGALTS GVHTFPAVLQ SSGLYSLSSV VTVPSSSLGT | |
| | QTYICNVNHK PSNTKVDKKV EPKSCDKTHT CPPCPAPELL | |
| | GGPSVFLFPP KPKDTLMISR TPEVTCVVVD VSHEDPEVKF | |
| | NWYVDGVEVH NAKTKPREEQ YNSTYRVVSV LTVLHQDWLN | |
| | GKEYKCKVSN KALPAPIEKT ISKAKGQPRE PQVYTLPPSR | |
| | DELTKNQVSL TCLVKGFYPS DIAVEWESNG QPENNYKTTP | |
| | PVLDSDGSFF LYSKLTVDKS RWQQGNVFSC SVMHEALHNH | |
| | YTQKSLSLSP GK | |
| 51 | DIQMTQSPST LSASVGDRVT ITCRASQSIS SWLAWYQQKP | LC sequence of RG6292 |
| | GKAPKLLIYK ASSLESGVPS RFSGSGSGTE FTLTISSLQP | |
| | DDFATYYCQQ YNIYPITFGG GTKVEIKRTV AAPSVFIFPP | |
| | SDEQLKSGTA SVVCLLNNFY PREAKVQWKV DNALQSGNSQ | |
| | ESVTEQDSKD STYSLSSTLT LSKADYEKHK VYACEVTHQG | |
| | LSSPVTKSEN RGEC | |
| 52 | YQCVQGYRALHRGP | amino acids 150-163 of |
| | | SEQ ID NO: 1 |
| 53 | SVCKMTHGKTRWTQPQLICTG | amino acids 166-186 of |
| | | SEQ ID NO: 1 |
| 54 | KEGTMLNCECKRGER | amino acids 42-56 of SEQ |
| | | ID NO: 1 |
| 55 | NSSHSSWDNQCQCTSSATR | amino acids 70-88 of SEQ |
| | | ID NO: 1 |
| 56 | YQCVQGYRA | amino acids 150-158 of |
| | | SEQ ID NO: 1 |
| 57 | RWTQP | amino acids 176-180 of |
| | | SEQ ID NO: 1 |
| 58 | SSWDNQCQCTS | amino acids 74-84 of SEQ |
| | | ID NO: 1 |
| 59 | NSSHSSWDNQCQCTS | amino acids 70-84 of SEQ |
| | | ID NO: 1 |

1. An anti-CD25 antibody for use in the treatment of acute myeloid leukemia (AML) or diffuse large B-cell lymphoma (DLBCL) in a subject.

2. The anti-CD25 antibody for use according to claim 1 wherein the anti-CD25 antibody inhibits less than 50% of the signalling of IL-2 via CD25 compared to IL-2 signalling in the absence of the antibody.

3. The anti-CD25 antibody for use according to claim 2 wherein the anti-CD25 antibody inhibits less than 25% of the signalling of IL-2 via CD25 compared to IL-2 signalling in the absence of the antibody.

4. The anti-CD25 antibody for use according to any one of claims 1 to 3 wherein the antibody is selected from the group consisting of:
(a) an antibody comprising:
a heavy chain variable region comprising a CDR-H1 comprising the amino acid sequence of any one of SEQ ID NOs: 2-5, a CDR-H2 comprising the amino acid sequence of any one of SEQ ID NOs: 6-11 and CDR-H3 comprising the amino acid sequence of SEQ ID NO: 12, and
a light chain variable region comprising a CDR-L1 comprising the amino acid sequence of SEQ ID NO: 13, a CDR-L2 comprising the amino acid sequence of SEQ ID NO: 14, and a CDR-L3 comprising the amino acid sequence of SEQ ID NO: 15;
(b) an antibody comprising:
a heavy chain variable region comprising a CDR-H1 comprising the amino acid sequence of SEQ ID NO: 23, a CDR-H2 comprising the amino acid sequence of SEQ ID NO: 24, a CDR-H3 comprising the amino acid sequence of SEQ ID NO: 25, and
a light chain variable region comprising a CDR-L1 comprising the amino acid sequence of SEQ ID NO: 26, a CDR-L2 comprising the amino acid sequence of SEQ ID NO: 27, and a CDR-L3 comprising the amino acid sequence of SEQ ID NO: 28; and
(c) an antibody comprising:
a heavy chain variable region comprising a CDR-H1 comprising the amino acid sequence of any one of SEQ ID NOs: 31-33, a CDR-H2 comprising the amino acid sequence of any one of SEQ ID NOs: 34-38, a CDR-H3 comprising the amino acid sequence of SEQ ID NO: 39, and
a light chain variable region comprising a CDR-L1 comprising the amino acid sequence of SEQ ID NO: 40, a CDR-L2 comprising the amino acid sequence of SEQ ID NO: 41, and a CDR-L3 comprising the amino acid sequence of SEQ ID NO: 42.

5. The anti-CD25 antibody for use according to any one of claims 1 to 4 wherein the antibody is RG6292.

6. The anti-CD25 antibody for use according to any one of claims 1 to 5 wherein the antibody binds to an epitope comprising at least one sequence selected from amino acids 150-158 of SEQ ID NO: 1, amino acids 176-180 of SEQ ID NO: 1, amino acids 42-56 of SEQ ID NO: 1 and amino acids 74-84 of SEQ ID NO: 1.

7. The anti-CD25 antibody for use according to claim 6 wherein the antibody binds to an epitope comprising amino acids 70-84 of SEQ ID NO: 1.

8. The anti-CD25 antibody for use according to any one of claims 1 to 7 wherein the antibody kills cancer, Treg, AML blast and/or PMBC cells.

9. The anti-CD25 antibody for use according to claim 8 wherein the antibody kills Treg cells and blast cells having an CD25 expression level of above about 900 CD25 molecules per cell.

10. The anti-CD25 antibody for use according to claim 9 wherein the antibody kills Treg cells and blast cells having an CD25 expression level of above about 1000 CD25 molecules per cell

11. The anti-CD25 antibody for use according to claim 10 wherein the antibody kills Treg cells and blast cells having an CD25 expression level in the range of from about 1000 to about 40000 CD25 molecules per cell.

12. The anti-CD25 antibody for use according to claim 11 wherein the antibody kills Treg cells and blast cells having an CD25 expression level in the range of from about 1000 to about 5000 CD25 molecules per cell.

13. The anti-CD25 antibody for use according to any one of claims 1 to 12 wherein the antibody induces ADCC activity.

14. The anti-CD25 antibody for use according to any one of claims 1 to 13 wherein the antibody is a monoclonal antibody.

15. The anti-CD25 antibody for use according to any one of claims 1 to 14 wherein the antibody is an IgG antibody.

16. The anti-CD25 antibody for use according to claim 15 wherein the antibody is an IgG1 antibody.

17. The anti-CD25 antibody for use according to any one of claims 1 to 16 wherein the antibody is a monospecific antibody.

18. The anti-CD25 antibody for use according to claim 17 wherein the antibody is a bivalent monospecific antibody.

19. The anti-CD25 antibody for use according to any one of claims 1 to 18 wherein the antibody is afucosylated.

20. The anti-CD25 antibody for use according to any one of claims 1 to 19 wherein the antibody is a human or humanised antibody.

21. The anti-CD25 antibody for use according to any one of claims 1 to 20, wherein the antibody induces a maximum reduction in CD16 expression on NK cells of 25%, when the antibody and NK cells are co-incubated with cells expressing 900 to 5000 CD25 molecules per cells.

22. The anti-CD25 antibody for use according to claim 21 wherein the NK cells are CD56dim NK cells.

23. The anti-CD25 antibody for use according to any one of claims 1 to 22 wherein the antibody is not conjugated to a further therapeutic agent.

24. The anti-CD25 antibody for use according to any one of claims 1 to 23 wherein the antibody is administered in combination with one or more further therapeutic agents.

25. The anti-CD25 antibody for use according to claim 24 wherein the one or more further therapeutic agents is selected from immune check point inhibitors, cancer vaccines, FLT3 inhibitors, BCL-2 inhibitors, IDH inhibitors, hypomethylating agents, anthracyclines and combinations thereof.

26. The anti-CD25 antibody for use according to claim 25 wherein the immune check point inhibitor is a PD-1 antagonist.

27. The anti-CD25 antibody for use according to claim 26 wherein the PD-1 antagonist is an anti-PD-1 antibody or an anti-PD-L1 antibody.

28. The anti-CD25 antibody for use according to claim 25. wherein the antibody is for use in combination with a BCL-2 inhibitor.

29. The anti-CD25 antibody for use according to claim 28 wherein the BCL-2 inhibitor is venetoclax.

30. The anti-CD25 antibody for use according to claim 25 wherein the hypomethylating agent is azacitidine.

31. The anti-CD25 antibody for use according to claim 25 wherein the one or more further therapeutic agents is a FLT3 Inhibitor.

32. The anti-CD25 antibody for according to claim 25, wherein the antibody is for use in combination with a BCL-2 inhibitor and a hypomethylating agent.

33. The anti-CD25 antibody for use according to any one of claims 1 to 23 wherein the antibody is for use as a monotherapy.

34. The anti-CD25 antibody for use according to any one of claims 1 to 33 wherein the CD25 expression level on tumor cells from the subject is at least about 900 CD25 molecules per cell.

35. The anti-CD25 antibody for use according to any one of claims 1 to 35 wherein the CD25 expression level on tumor cells from the subject is in the range of from about 900 to about 5000 CD25 molecules per cell.

36. A combination of an anti-CD25 antibody as defined in any one of claims 1 to 23 and one or more further therapeutic agent, for use in the treatment of acute myeloid leukemia or diffuse large B-cell lymphoma, wherein the anti-CD25 antibody and the further therapeutic agent are for separate, simultaneous or sequential administration.

37. The combination for use as claimed in claim 36, wherein the one or more further therapeutic agent is as defined in any one of claims 25 to 32.

38. A method of treating acute myeloid leukemia (AML) or diffuse large B-cell lymphoma (DLBCL) in a subject comprising administrating to the subject an effective amount of an anti-CD25 antibody.

39. The method according to claim 38 wherein the antibody is as defined in any one of claims 1 to 23.

40. The method according to claim 38 or claim 39, wherein the anti-CD25 antibody is administered as a monotherapy.

41. The method of claim 38 or 39, wherein the method further comprises administration of one or more further therapeutic agents, for example a therapeutic agent as defined in any one of claims 25 to 32.

42. The method of any one of claims 38 to 41, wherein the CD25 expression level on tumor cells from the subject is at least about 900 CD25 molecules per cell.

43. The method of claim 42, wherein the CD25 expression level on tumor cells from the subject is in the range of from about 900 to about 5000 CD25 molecules per cell.

44. Use of anti-CD25 antibody in the manufacture of a medicament for the treatment of acute myeloid leukemia or diffuse large B-cell lymphoma.

45. The use according to claim 44 wherein the antibody is as defined in any one of claims 1 to 23.

46. The use according to claim 44 or claim 45, wherein the anti-CD25 antibody is for use as a monotherapy.

47. The use according to claim 44 or 45, wherein the use is in combination with one or more further therapeutic agents, for example a therapeutic agent as defined in any one of claims 25 to 32.

48. The use of a combination of an anti-CD25 antibody and a further therapeutic agent in the manufacture of a medicament for the treatment of acute myeloid leukemia or diffuse large B-cell lymphoma.

49. The use according to claim 48, wherein the antibody is as defined in any one of claims 1 to 23.

50. The use according to claim 48 or 49, wherein the further therapeutic agent is as defined in any one of claims 25 to 32.

51. A method of selecting a patient having acute myeloid leukemia for treatment with an anti-CD25 antibody, the method comprising determining the expression level of CD25 on target cells in a sample from the patient, wherein if the cells have an expression level above about 900 CD25 molecules per cell the patient is suitable for treatment with the antibody.

52. The method according to claim 51 wherein the sample is a bone marrow sample from the patient.

53. The method according to any one of claim 51 or 52 wherein the target cells are blast and/or Treg cells.

54. The method according to any one of claims 51 to 53 wherein the expression levels of CD25are determined by flow cytometry.

55. The method according to any one of claims 51 to 54, further comprising administering the patient with the anti-CD25 antibody, if they are determined to have expression levels of above 900 CD25 molecules per cell.

56. The method according to claim 55 wherein the anti-CD25 antibody is as defined in any one of claims 1 to 23.

57. A method of selecting a patient having AML for treatment with an anti-CD25 antibody, the method comprising determining the presence or absence of an FLT3-ITD mutation in a sample from the patient, wherein if the mutation is present in the sample the patient is suitable for treatment with the antibody.

58. The method according to claim 57 wherein the method further comprises administering the anti-CD25 antibody to the patient, if they are determined to have an FLT3-ITD mutation.

59. A method of predicting the response of an AML patient to treatment with an anti-CD25 antibody, the method comprising determining the presence or absence of an FLT3-ITD mutation in a sample from the patient, wherein the presence of the mutation in the sample is indicative for a patient who will respond to treatment with the anti-CD25antibody.

60. A method of treating acute myeloid leukemia (AML) in a subject comprising administrating to the subject an effective amount of an anti-CD25 antibody, wherein the subject comprises the presence of an FLT3-ITD mutation.

61. The method according to claim 60, wherein the method further comprising determining the presence of an FLT3-ITD mutation in a sample from a patient with AML.

62. The method according to any one of claim 57 to 59 or 61 wherein the sample is a blood or bone marrow sample from the patient.

63. The method according to claims 57 to 59, or 61 to 62 wherein the presence or absence of the mutation is determined by a method selected from the group of DNA sequencing and mutation screening technology.

64. A method for preventing or reducing the risk of relapse in an AML patient, the method comprising administering an anti-CD25 antibody to the patient.

65. The method according to claim 64 wherein the method further comprises administration one or more further therapeutic agents.

66. The method according to claim 65, wherein the one or more therapeutic agent is as defined in any one of claims 25 to 32.

67. A method for treating AML in a patient who has undergone BCL-2 inhibitor-hypomethylating agent combination treatment, the method comprising administering an anti-CD25 antibody to the patient.

68. The method according to any one of claims 57 to 67, wherein the anti-CD25 antibody is as defined in any one of claims 1 to 23.


SEQUENCE LISTING (59 sequences)

SEQ ID NO: 1 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 2 - protein, 9 aa, Homo sapiens
GTFSSLAIS

SEQ ID NO: 3 - protein, 9 aa, Homo sapiens
GTFSSLAIT

SEQ ID NO: 4 - protein, 9 aa, Homo sapiens
GTFSALAIS

SEQ ID NO: 5 - protein, 9 aa, Homo sapiens
GTFSSLAIF

SEQ ID NO: 6 - protein, 17 aa, Homo sapiens
GIIPIFGTANYAQKFQG

SEQ ID NO: 7 - protein, 17 aa, Homo sapiens
AIIPVFGTASYAQKFQG

SEQ ID NO: 8 - protein, 17 aa, Homo sapiens
GIIPIFGDASYAQKFQG

SEQ ID NO: 9 - protein, 17 aa, Homo sapiens
GIIPIFGDANYAQKLQG

SEQ ID NO: 10 - protein, 17 aa, Homo sapiens
GIIPLFGRANYAQKFQG

SEQ ID NO: 11 - protein, 17 aa, Homo sapiens
GIIPVFGQANYAQKFQG

SEQ ID NO: 12 - protein, 15 aa, Homo sapiens
ARGGSVSGTLVDFDI

SEQ ID NO: 13 - protein, 11 aa, Homo sapiens
RASQSISSWLA

SEQ ID NO: 14 - protein, 7 aa, Homo sapiens
KASSLES

SEQ ID NO: 15 - protein, 9 aa, Homo sapiens
QQYNIYPIT

SEQ ID NO: 16 - protein, 122 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSS

SEQ ID NO: 17 - protein, 122 aa, Homo sapiens
QVQLVQSGAEVKKPGSSVKVSCKASGGTFSSLAISWVRQAPGQGLEWMGAIIPVFGTASYAQKFQGRVTITADESTSTAYMELSSLRSEDTAVYYCARGGSVSGTLVDFDIWGQGTMVTVSS

SEQ ID NO: 18 - protein, 122 aa, Homo sapiens
QVQLVQSGAEVKKPGSSVKVSCKASGGTFSSLAITWVRQAPGQGLEWMGGIIPIFGDASYAQKFQGRVTITADESTSTAYMELSSLRSEDTAVYYCARGGSVSGTLVDFDIWGQGTMVTVSS

SEQ ID NO: 19 - protein, 122 aa, Homo sapiens
QVQLVQSGAEVKKPGSSVKVSCKASGGTFSSLAISWVRQAPGQGLEWMGGIIPIFGDANYAQKLQGRVTMTTDTSTSTAYMELRSLRSDDTAVYYCARGGSVSGTLVDFDIWGQGTMVTVSS

SEQ ID NO: 20 - protein, 122 aa, Homo sapiens
QVQLVQSGAEVKKPGSSVKVSCKASGGTFSALAISWVRQAPGQGLEWMGGIIPLFGRANYAQKFQGRVTITADESTSTAYMELSSLRSEDTAVYYCARGGSVSGTLVDFDIWGQGTMVTVSS

SEQ ID NO: 21 - protein, 122 aa, Homo sapiens
QVQLVQSGAEVKKPGSSVKVSCKASGGTFSSLAIFWVRQAPGQGLEWMGGIIPVFGQANYAQKFQGRVTITVDESTSTAYMELSSLRSEDTAVYYCARGGSVSGTLVDFDIWGQGTMVTVSS

SEQ ID NO: 22 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIK

SEQ ID NO: 23 - protein, 9 aa, Homo sapiens
YTFTSYYMH

SEQ ID NO: 24 - protein, 17 aa, Homo sapiens
IINPSGGSTSYAQKFQG

SEQ ID NO: 25 - protein, 15 aa, Homo sapiens
ARGGAEYIPAEYFQH

SEQ ID NO: 26 - protein, 11 aa, Homo sapiens
RASQSVSSYLA

SEQ ID NO: 27 - protein, 7 aa, Homo sapiens
DASNRAT

SEQ ID NO: 28 - protein, 8 aa, Homo sapiens
QQRPFLPT

SEQ ID NO: 29 - protein, 122 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGAEYIPAEYFQHWGQGTLVTVSS

SEQ ID NO: 30 - protein, 106 aa, Homo sapiens
EIVMTQSPATLSLSPGERATLSCRASQSVSSYLAWYQQKPGQAPRLLIYDASNRATGIPARFSGSGSGTDFTLTISSLEPEDFAVYYCQQRPFLPTFGGGTKVEIK

SEQ ID NO: 31 - protein, 9 aa, Homo sapiens
FTFSSYGMH

SEQ ID NO: 32 - protein, 9 aa, Homo sapiens
FTFASYGMH

SEQ ID NO: 33 - protein, 9 aa, Homo sapiens
FTFPSYGMH

SEQ ID NO: 34 - protein, 17 aa, Homo sapiens [sequence omitted]

SEQ ID NO: 35 - protein, 17 aa, Homo sapiens
VIWYDASTKYYADSVKG

SEQ ID NO: 36 - protein, 17 aa, Homo sapiens
VIWYDAINKYYADSVKG

SEQ ID NO: 37 - protein, 17 aa, Homo sapiens
VIWYDAVNKYYADSVKG

SEQ ID NO: 38 - protein, 17 aa, Homo sapiens
VIWYDALNKYYADSVKG

SEQ ID NO: 39 - protein, 14 aa, Homo sapiens
ARDLGYGDYAAHDY

SEQ ID NO: 40 - protein, 11 aa, Homo sapiens
RASQSISSWLA

SEQ ID NO: 41 - protein, 7 aa, Homo sapiens
KASSLES

SEQ ID NO: 42 - protein, 9 aa, Homo sapiens
QQHNTHPYT

SEQ ID NO: 43 - protein, 121 aa, Homo sapiens
QVQLVESGGGVVQPGRSLRLSCAASGFTFSSYGMHWVRQAPGKGLEWVAVIWYDGSNKGYADSVKGRFTISRDNSKNTLYLQMNSLRAEDTAVYYCARDLGYGDYAAHDYWGQGTLVTVSS

SEQ ID NO: 44 - protein, 121 aa, Homo sapiens
EVQLVESGGGLVKPGGSLRLSCAASGFTFASYGMHWVRQAPGKGLEWVAVIWYDASTKYYADSVKGRFTISRDNSKNTLYLQMNSLRAEDTAVYYCARDLGYGDYAAHDYWGQGTLVTVSS

SEQ ID NO: 45 - protein, 121 aa, Homo sapiens
QVQLVESGGGLVQPGGSLRLSCAASGFTFPSYGMHWVRQAPGKGLEWVAVIWYDAINKYYADSVKGRFTISRDNSKNTLYLQMNSLRAEDTAVYYCARDLGYGDYAAHDYWGQGTLVTVSS

SEQ ID NO: 46 - protein, 121 aa, Homo sapiens
QVQLVESGGGVVQPGRSLRLSCAASGFTFPSYGMHWVRQAPGKGLEWVAVIWYDAVNKYYADSVKGRFTISRDNSKNTLYLQMNSLRAEDTAVYYCARDLGYGDYAAHDYWGQGTLVTVSS

SEQ ID NO: 47 - protein, 121 aa, Homo sapiens
QVQLVESGGGVVQPGRSLRLSCAASGFTFPSYGMHWVRQAPGKGLEWVAVIWYDASTKYYADSVKGRFTISRDNSKNTLYLQMNSLRAEDTAVYYCARDLGYGDYAAHDYWGQGTLVTVSS

SEQ ID NO: 48 - protein, 121 aa, Homo sapiens
EVQLLESGGGLVQPGGSLRLSCAASGFTFPSYGMHWVRQAPGKGLEWVAVIWYDALNKYYADSVKGRFTISRDNSKNTLYLQMNSLRAEDTAVYYCARDLGYGDYAAHDYWGQGTLVTVSS

SEQ ID NO: 49 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQHNTHPYTFGGGTKVEIK

SEQ ID NO: 50 - protein, 452 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 14 aa, Homo sapiens
YQCVQGYRALHRGP

SEQ ID NO: 53 - protein, 21 aa, Homo sapiens
SVCKMTHGKTRWTQPQLICTG

SEQ ID NO: 54 - protein, 15 aa, Homo sapiens
KEGTMLNCECKRGFR

SEQ ID NO: 55 - protein, 19 aa, Homo sapiens
NSSHSSWDNQCQCTSSATR

SEQ ID NO: 56 - protein, 9 aa, Homo sapiens
YQCVQGYRA

SEQ ID NO: 57 - protein, 5 aa, Homo sapiens
RWTQP

SEQ ID NO: 58 - protein, 11 aa, Homo sapiens
SSWDNQCQCTS

SEQ ID NO: 59 - protein, 15 aa, Homo sapiens
NSSHSSWDNQCQCTS